(19) United States
(12) Patent Application Publication
(10) Pub. No.: US 2026/0041503 A1
(43) Pub. Date: Feb. 12, 2026

(54) ROBOTIC MEDICAL SYSTEMS AND METHODS

(71) Applicant: Remedy Robotics, Inc., San Francisco, CA (US)

(72) Inventors: Jake Anthony Sganga, Mill Valley, CA (US); David James Bell, San Francisco, CA (US)

(21) Appl. No.: 19/294,661

(22) Filed: Aug. 8, 2025

Related U.S. Application Data
(60) Provisional application No. 63/681,361, filed on Aug. 9, 2024.
(60) Provisional application No. 63/681,372, filed on Aug. 9, 2024.
(60) Provisional application No. 63/681,356, filed on Aug. 9, 2024.

(51) Int. Cl. A61B 34/30; A61B 17/00

(52) U.S. Cl. CPC A61B 34/30; A61B 17/00; A61B 2017/00973; A61B 2034/301

(57) ABSTRACT

Robotic medical systems and methods are described. A robotic medical system can include an enable input that must be activated in order for the robotic medical system to act on user commands to manipulate an endovascular instrument. When the enable input is activated and no user commands to manipulate the endovascular instrument are provided, the robotic medical system can perform automated background tasks. A robotic medical system can include processes for mitigation slack or buckling of an endovascular instrument.

### PRIORITY APPLICATIONS

This application claims priority to U.S. Provisional Application No. 63/681,361, filed Aug. 9, 2024, U.S. Provisional Application No. 63/681,372, filed Aug. 9, 2024, and U.S. Provisional Application No. 63/681,356, filed Aug. 9, 2024, each of which are incorporated herein by reference. Any and all applications for which a foreign or domestic priority claim is identified in the Application Data Sheet as filed with the present application are hereby incorporated by reference under 37 CFR 1.57.

### BACKGROUND

### Field

Some embodiments of the present disclosure are directed to robotic medical systems and methods. In particular, some embodiments of the present disclosure are directed to robotic medical systems and methods configured to facilitate endovascular procedures.

### Description

Endovascular medical procedures are common. During an endovascular procedure, a tool or medical instrument that is generally configured as a thin, flexible, elongate body is inserted into and navigated through a lumen or other cavity of the body.

In some instances, the tools or medical instruments are articulable or controllable, for example, using one or more pull wires, to allow an operator to navigate the tool or medical instrument within the body. Such navigation is often accomplished through deflection (for example, bending) of the distal tip of the tool or medical instrument.

Some tools or medical instruments are configured for manual control, for example, using knobs or levers mounted on a proximally located handle of the tool or medical instrument. In other instances, the tools or medical instruments can be configured for robotic control, for example, control by a robotic medical system. In some embodiments, an operator can use the robotic medical system (for example, a controller, user interface, and/or the like) to robotically control the tool or medical instrument.

### SUMMARY

For purposes of this summary, certain aspects, advantages, and novel features of the invention are described herein. It is to be understood that not all such advantages necessarily may be achieved in accordance with any particular embodiment of the inventions described herein. Thus, for example, those skilled in the art will recognize that the inventions described herein may be embodied or conducted in a manner that achieves one advantage or group of advantages as taught herein without necessarily achieving other advantages as may be taught or suggested herein.

In some aspects, the techniques described herein relate to a robotic medical system, including: an endovascular robot configured at robotically manipulate one or more endovascular instruments insertable into a vasculature of a patient during an endovascular procedure; a user control station configured to receive: robot control user inputs, provided by a user, for causing the endovascular robot to robotically manipulate the one or more endovascular instruments, and an enable input provided by the user; and a robot control unit in communication with the user control station and the endovascular robot, the robot control unit including at least one processor and at least one computer memory, the at least one computer memory including computer-readable instructions that, when executed by the processor, cause the processor to: cause the endovascular robot to manipulate the one or more endovascular instruments according to the robot control user inputs received from the user control station only when an enable input is also received from the user control station; and cause the endovascular robot to perform automated background tasks when the enable input is received from the user control station and no robot control user inputs are received from the user control station.

In some aspects, the techniques described herein relate to a robotic medical system, wherein the computer-readable instructions, when executed by the processor, further cause the processor to disregard the robot control user inputs received from the user control station when no enable input is received from the user control station.

In some aspects, the techniques described herein relate to a robotic medical system, wherein the automated background tasks include performing a slack removal process configured to remove slack from the one or more endovascular instruments.

In some aspects, the techniques described herein relate to a robotic medical system, wherein the slack removal process includes causing retraction, by a proximally located helm of the endovascular robot, of the one or more endovascular instruments until a force or torque measured at a distally located helm of the endovascular robot exceeds a predetermined force or torque threshold.

In some aspects, the techniques described herein relate to a robotic medical system, wherein the predetermined force or torque threshold is determined so as to be indicative of the one or more endovascular instruments going taut between the proximally located helm and the distally located helm.

In some aspects, the techniques described herein relate to a robotic medical system, wherein the automated background tasks include performing a buckling removal process configured to remove buckling from the one or more endovascular instruments.

In some aspects, the techniques described herein relate to a robotic medical system, wherein the buckling removal process includes causing, with the endovascular robot, retraction of the one or more endovascular instruments until a computer vision analysis of a medical image showing a distal tip of the one more endovascular instruments determines that the distal tip moves in a backwards direction relative to a current heading of the distal tip by a distance that exceeds a predetermined threshold.

In some aspects, the techniques described herein relate to a robotic medical system, wherein the backwards direction relative to the current heading includes an arcuate area.

In some aspects, the techniques described herein relate to a robotic medical system, wherein the automated background tasks include adjusting a position of a moveable helm of the endovascular robot in a manner that does not cause movement of a distal tip of the one or more endovascular instruments.

In some aspects, the techniques described herein relate to a robotic medical system, wherein user control station includes a foot pedal, and the enable input is provided through the foot pedal.

In some aspects, the techniques described herein relate to a robotic medical method including: receiving, from a user control station one or more of: robot control user inputs, provided by a user, for causing an endovascular robot to robotically manipulate one or more endovascular instruments, or an enable input provided by the user; causing the endovascular robot to manipulate the one or more endovascular instruments according to the robot control user inputs received from the user control station only when an enable input is also received from the user control station; and causing the endovascular robot to perform automated background tasks when the enable input is received from the user control station and no robot control user inputs are received from the user control station.

In some aspects, the techniques described herein relate to a robotic medical method, further including disregarding the robot control user inputs received from the user control station when no enable input is received from the user control station.

In some aspects, the techniques described herein relate to a robotic medical method, wherein the automated background tasks include performing a slack removal process configured to remove slack from the one or more endovascular instruments.

In some aspects, the techniques described herein relate to a robotic medical method, wherein the slack removal process includes causing retraction, by a proximally located helm of the endovascular robot, of the one or more endovascular instruments until a force or torque measured at a distally located helm of the endovascular robot exceeds a predetermined force or torque threshold.

In some aspects, the techniques described herein relate to a robotic medical method, wherein the predetermined force or torque threshold is determined so as to be indicative of the one or more endovascular instruments going taut between the proximally located helm and the distally located helm.

In some aspects, the techniques described herein relate to a robotic medical method, wherein the automated background tasks include performing a buckling removal process configured to remove buckling from the one or more endovascular instruments.

In some aspects, the techniques described herein relate to a robotic medical method, wherein the buckling removal process includes causing, with the endovascular robot, retraction of the one or more endovascular instruments until a computer vision analysis of a medical image showing a distal tip of the one more endovascular instruments determines that the distal tip moves in a backwards direction relative to a current heading of the distal tip by a distance that exceeds a predetermined threshold.

In some aspects, the techniques described herein relate to a robotic medical method, wherein the backwards direction relative to the current heading includes an arcuate area.

In some aspects, the techniques described herein relate to a robotic medical method, wherein the automated background tasks include adjusting a position of a moveable helm of the endovascular robot in a manner that does not cause movement of a distal tip of the one or more endovascular instruments.

In some aspects, the techniques described herein relate to a robotic medical method, wherein user control station includes a foot pedal, and the method further includes receiving the enable input from the foot pedal.

In some aspects, the techniques described herein relate to a robotic medical system, including: a rail extending between a distal end and a proximal end; a distally positioned helm mounted on the rail, the distally positioned helm engaged with a body of an endovascular instrument extending through the distally positioned helm, the distally positioned helm configured to cause insertion, in a distal direction, and retraction, in a proximal direction, of the endovascular instrument, the proximally positioned helm further including a torque or force sensor; a proximally positioned helm mounted on the rail, the proximally positioned helm engaged with the body of the endovascular instrument, the proximally positioned helm configured to cause insertion, in the distal direction, and retraction, in the proximal direction, of the endovascular instrument; a robot control unit in communication with the distally positioned helm and the proximally positioned helm, the robot control unit including at least one processor and at least one computer memory, the at least one computer memory including computer-readable instructions that, when executed by the processor, cause the processor to remove slack in the endovascular instrument between the distally positioned helm and the proximally positioned helm by: causing the proximally positioned helm to retract, in the proximal direction, the endovascular instrument, and based on receipt of a measured torque or force from the force sensor of the distally positioned helm exceeding a predetermined threshold, causing the proximally positioned helm to stop retracting the endovascular instrument, wherein the predetermined threshold is indicative of a force or torque experienced when the endovascular instrument is taut between the distally positioned helm and the proximally positioned helm.

In some aspects, the techniques described herein relate to a robotic medical system, wherein the distally positioned helm includes a stationary helm fixedly positioned at a distal end of the rail.

In some aspects, the techniques described herein relate to a robotic medical system, wherein the distally positioned helm includes a moveable helm configured to move linearly along the rail.

In some aspects, the techniques described herein relate to a robotic medical system, wherein the proximally positioned helm includes a moveable helm configured to move linearly along the rail.

In some aspects, the techniques described herein relate to a robotic medical system, wherein a first removable tool is received within a recess of the distally positioned helm, the first removable tool engaged with the endovascular instrument.

In some aspects, the techniques described herein relate to a robotic medical system, wherein a second removable tool is received within a recess of the proximally positioned helm, the second removable tool engaged with the endovascular instrument.

In some aspects, the techniques described herein relate to a robotic medical system, wherein the proximally positioned helm includes: a pair of rollers engaged with the endovascular instrument; at least one motor configured to cause rotation of the pair of rollers, wherein rotation of the pair of rollers causes insertion or retraction of the endovascular instrument; and the force or torque sensor is coupled to one of the rollers or the at least one motor.

In some aspects, the techniques described herein relate to a robotic medical system, wherein the proximally positioned helm is configured to move in a proximal direction along the rail to cause retraction of the endovascular instrument.

In some aspects, the techniques described herein relate to a robotic medical system, wherein the endovascular instrument includes a catheter.

In some aspects, the techniques described herein relate to a robotic medical system, wherein the endovascular instrument includes a wire.

In some aspects, the techniques described herein relate to a robotic medical method, including: causing a proximally positioned helm to retract, in a proximal direction, an endovascular instrument, wherein: the proximally positioned helm is mounted on a rail that extends between a distal end and a proximal end, the proximally positioned helm is engaged with a body of the endovascular instrument, and the proximally positioned helm is configured to cause insertion, in the distal direction, and retraction, in the proximal direction, of the endovascular instrument, and based on receipt of a measured torque or force from a force sensor of a distally positioned helm exceeding a predetermined threshold, causing the proximally positioned helm to stop retracting the endovascular instrument, wherein: the distally positioned helm is mounted on the rail, the distally positioned helm is engaged with the body of the endovascular instrument extending through the distally positioned helm, the distally positioned helm is configured to cause insertion, in a distal direction, and retraction, in a proximal direction, of the endovascular instrument, and the proximally positioned helm includes the torque or force sensor; wherein the predetermined threshold is indicative of a force or torque experienced when the endovascular instrument is taut between the distally positioned helm and the proximally positioned helm.

In some aspects, the techniques described herein relate to a robotic medical method, wherein the distally positioned helm includes a stationary helm fixedly positioned at a distal end of the rail.

In some aspects, the techniques described herein relate to a robotic medical method, wherein the distally positioned helm includes a moveable helm configured to move linearly along the rail.

In some aspects, the techniques described herein relate to a robotic medical method, wherein the proximally positioned helm includes a moveable helm configured to move linearly along the rail.

In some aspects, the techniques described herein relate to a robotic medical method, wherein a first removable tool is received within a recess of the distally positioned helm, the first removable tool engaged with the endovascular instrument.

In some aspects, the techniques described herein relate to a robotic medical method, wherein a second removable tool is received within a recess of the proximally positioned helm, the second removable tool engaged with the endovascular instrument.

In some aspects, the techniques described herein relate to a robotic medical method, wherein the proximally positioned helm includes: a pair of rollers engaged with the endovascular instrument; at least one motor configured to cause rotation of the pair of rollers, wherein rotation of the pair of rollers causes insertion or retraction of the endovascular instrument; and the force or torque sensor is coupled to one of the rollers or the at least one motor.

In some aspects, the techniques described herein relate to a robotic medical method, wherein the proximally positioned helm is configured to move in a proximal direction along the rail to cause retraction of the endovascular instrument.

In some aspects, the techniques described herein relate to a robotic medical method, wherein the endovascular instrument includes a catheter.

In some aspects, the techniques described herein relate to a robotic medical method, wherein the endovascular instrument includes a wire.

In some aspects, the techniques described herein relate to a robotic medical system, including: an endovascular robot configured at robotically manipulate an endovascular instrument insertable into a vasculature of a patient during an endovascular procedure; a robot control unit in communication with the endovascular robot, the robot control unit including at least one processor and at least one computer memory, the at least one computer memory including computer-readable instructions that, when executed by the processor, cause the processor to remove buckling in the endovascular instrument by: causing the endovascular robot to retract, in a proximal direction, the endovascular instrument, and analyze a medical image received from a medical imaging device to determine that a distal tip of the endovascular instrument moves in a backwards direction; and based on determining that the distal tip of the endovascular instrument has moved in the backwards direction by a distance that exceeds a predetermined threshold, causing the endovascular robot to stop retracting the endovascular instrument.

In some aspects, the techniques described herein relate to a robotic medical system, wherein the computer-readable instructions, when executed by the processor, further cause the processor to determine a current heading of the distal tip of the endovascular instrument in the medical image.

In some aspects, the techniques described herein relate to a robotic medical system, wherein the backwards direction is defined with respect to current heading of the distal tip of the endovascular instrument in the medical image.

In some aspects, the techniques described herein relate to a robotic medical system, wherein the processor is configured to determine backwards direction based on determining that the distal tip moves into an arcuate area defined opposite the current heading.

In some aspects, the techniques described herein relate to a robotic medical system, wherein the endovascular instrument includes a catheter or a wire.

In some aspects, the techniques described herein relate to a robotic medical method, including: causing an endovascular robot to retract, in a proximal direction, an endovascular instrument, and analyzing a medical image received from a medical imaging device to determine that a distal tip of the endovascular instrument moves in a backwards direction; and based on determining that the distal tip of the endovascular instrument has moved in the backwards direction by a distance that exceeds a predetermined threshold, causing the endovascular robot to stop retracting the endovascular instrument.

In some aspects, the techniques described herein relate to a robotic medical method, further including determining a current heading of the distal tip of the endovascular instrument in the medical image.

In some aspects, the techniques described herein relate to a robotic medical method, wherein the backwards direction is defined with respect to current heading of the distal tip of the endovascular instrument in the medical image.

In some aspects, the techniques described herein relate to a robotic medical method, wherein to determining that the distal tip has moved in the backwards direction is based on determining that the distal tip moves into an arcuate area defined opposite the current heading.

In some aspects, the techniques described herein relate to a robotic medical method, wherein the endovascular instrument includes a catheter or a wire.

Although several configurations, examples, and illustrations are disclosed below, it will be understood by those of ordinary skill in the art that the disclosure extends beyond the specifically disclosed configurations, examples, and illustrations and includes other uses of the disclosure. Configurations are described with reference to the accompanying figures, wherein like numerals refer to like elements throughout. The terminology used in the description presented herein is not intended to be interpreted in any limited or restrictive manner simply because it is being used in conjunction with a detailed description of some specific configurations of the disclosure. In addition, configurations can comprise several novel and inventive features. No single feature is solely responsible for its desirable attributes or is essential to practicing the disclosure herein described.

### BRIEF DESCRIPTION OF THE DRAWINGS

Various features will now be described with reference to the following drawings. Throughout the drawings, reference numbers may be re-used to indicate correspondence between referenced elements. The drawings are provided to illustrate examples described herein and are not intended to limit the scope of the disclosure.

FIG. 1 illustrates a perspective view of an embodiment of some components of a robotic medical system that includes an endovascular robot configured to facilitate endovascular procedures.

FIG. 2 illustrates a perspective view of an embodiment of the endovascular robot of the robotic medical system of FIG. 1 shown with no tools attached thereto.

FIG. 3 is a perspective view of an embodiment of a bed mount useable to attach and position an endovascular robot to a bed in the robotic medical system of FIG. 1.

FIGS. 4A and 4B are example fluoroscopic images of a catheter configured with radio-opaque fiducials that allow determination of catheter pose using a computer vision algorithm.

FIG. 5A illustrates how the example arrangement radio-opaque fiducials shown in FIGS. 4A and 4B positioned on the distal end of the catheter may provide a unique appearance at different inclination and roll angles.

FIG. 5B illustrates another example of how the radio-opaque fiducials shown in FIGS. 4A and 4B positioned on the distal end of the catheter may provide a unique appearance at roll angles.

FIG. 6A illustrates examples of projections of three-dimensional generated catheters onto real world two-dimensional X-ray images.

FIGS. 6B-6C illustrate an example prediction of a neural network for predicting the position of a body of a catheter.

FIG. 6D illustrates an example, in which a neural network has identified the catheter within the image, superimposed its estimated centerline onto the image, and highlighted the catheter.

FIG. 6E illustrates an example in which the distal tip position and heading angle have been determined and the image has been updated to include a highlight identifying the position and an arrow indicating the heading.

FIGS. 7A-7D illustrate an embodiment of a graphical user interface for providing image space control of a medical instrument.

FIG. 8 illustrates an embodiment of a user control station that can be used to control the robotic medical system.

FIG. 9 illustrates a simplified schematic version of the robotic medical system, illustrating certain features relating to a background task and safety enable input.

FIG. 10 is a flow chart illustrating a method for operating a robotic medical system that includes a background task and safety enable input.

FIG. 11 illustrates a proposed architecture for facilitating a robotic medical system having a background task and safety enable input.

FIG. 12A illustrates an example showing slack of an endovascular tool between a distal helm and a moveable helm located proximally to the distal helm.

FIG. 12B illustrates an example showing removal of the slack of the endovascular tool between the distal helm and the moveable helm located proximally to the distal helm.

FIG. 13 illustrates an example method for removing slack in a robotic medical system.

FIG. 14A illustrates an example X-ray image of a distal tip of an endovascular tool positioned within a vessel wherein the endovascular tool exhibits a buckling.

FIG. 14B illustrates the same endovascular tool within the vessel after a degree of the buckling has been removed.

FIG. 15 illustrates an example method for removing buckling in a robotic medical system.

FIG. 16 illustrates that an endovascular instrument can be retracted until the distal tip moves into an arcuate area defined in a direction opposite the current heading of the endovascular instrument to remove buckling.

### DETAILED DESCRIPTION

In the following description, various examples will be described. For purposes of explanation, specific configurations and details are set forth in order to provide a thorough understanding of the examples. However, it will also be apparent to one skilled in the art that the examples may be practiced without specific details. Further, well-known features may be omitted or simplified in order not to obscure the examples being described.

### Endovascular Procedures

Endovascular procedures are minimally invasive medical techniques used to treat medical conditions that occur inside blood vessels (arteries or veins) using catheters and other tools that are inserted through small incisions, usually in the groin or wrist. In general, an endovascular procedure involves inserting a catheter (e.g., a thin, flexible tube) or other tool into a blood vessel. The catheter or tool is guided through the vasculature to a target area typically using imaging techniques such fluoroscopy (real-time X-ray). Once the catheter or tool is positioned at the target site, tools or other devices (such as balloons, stents, or coils) can be passed through the catheter to treat the issue.

Traditional methods for navigating through the vasculature involve manually advancing and/or rotating a simple catheter over a guidewire into the correct location. Such manual techniques require a high degree of skill and are often imprecise. This lack of precision can result in substantial vessel wall trauma, higher risk of complications, and longer procedure durations that are more difficult to perform.

Common endovascular procedures include, for example, angioplasty (widening narrowed or blocked arteries with a balloon), stenting (placing a small mesh tube (stent) to keep a vessel open), aneurysm repair (placing a stent graft inside an aneurysm to prevent rupture (e.g., endovascular aneurysm repair (EVAR) for abdominal aortic aneurysm)), thrombectomy/embolectomy (removing a blood clot from a vessel), embolization (blocking blood flow to abnormal vessels or tumors), and carotid artery stenting (used to treat narrowing of carotid arteries in the neck (often to prevent stroke)). The systems and methods described herein can be useful in performing these and other endovascular procedures in an improved manner. Endovascular procedures can provide many benefits, including being less invasive than open surgery, requiring smaller incisions and scars, reducing risk of infection, requiring shorter hospital stays, and/or allowing for faster recovery, among others. Endovascular procedures are commonly performed by interventional radiologists, vascular surgeons, or interventional cardiologists, depending on the condition being treated.

Cardiovascular disease (e.g., stroke, heart attack, blood clots, pulmonary embolism, and the like) is the number one cause of death and disability in the United States and around the world. Much of this can be prevented with better access to endovascular intervention (EI). For example, patient outcomes following stroke are directly related to the time it takes to undergo EI. Studies have shown that a person's chance of a return to functional independence drops by greater than 12% for every hour EI is delayed. This is particularly problematic because, due to a lack of expertise, EI is currently only available in about 2% of United States hospitals. The median time to transfer to one of these hospitals is greater than 3 hours. Devastatingly, this means that two thirds of all large vessel occlusion (LVO) stroke victims in the US lack timely access to EI.

LVO is the most significant cause of adult disability in the world. The only proven way to avoid severe post-stroke disability is to undergo an endovascular procedure called mechanical thrombectomy (MT) as quickly as possible. Despite the fact that the facilities required to perform MT are available in over 50% of United States hospitals, due to a lack of specialist expertise, MT is only offered in 2% of hospitals. This means that for patients who present to one of the 98% of hospitals that do not offer MT, treatment is either significantly delayed or not offered at all because timely transfer is not feasible. Because stroke is such a time-sensitive condition, any delay can lead to permanent disabilities or even death.

MT is the current gold standard of care for stroke treatment. MT involves physically retrieving a clot (via stent or suction) from within a blood vessel to restore blood flow in the brain. During MT, devices are threaded through the patient's vasculature starting in the femoral artery (groin) or radial artery (arm). The devices are navigated to the site of the clot using angiography (real-time x-ray images of the blood vessels with the injection of radio-opaque contrast agent).

As noted previously, EIs, like mechanical thrombectomy, are typically performed manually and require an onsite specialist. This leads to the problems associated with access to EI described above. However, endovascular treatment is quite unlike other surgery, which makes remotely supervised and autonomous intervention actually feasible. The systems and methods described here enable stroke patients around the world to undergo mechanical thrombectomy and other endovascular procedures as quickly and as safely as possible. Thus, the systems and methods described herein can improve patient outcomes by increasing access to EI as well as providing EI in a safer manner than manual procedures.

### Robotic Medical System

As described herein, endovascular procedures can be provided using a robotic medical system. Use of a robotic medical system for endovascular procedures can both improve patient access to endovascular procedures and facilitate performance of endovascular procedures in a safe, timely, and effective manner.

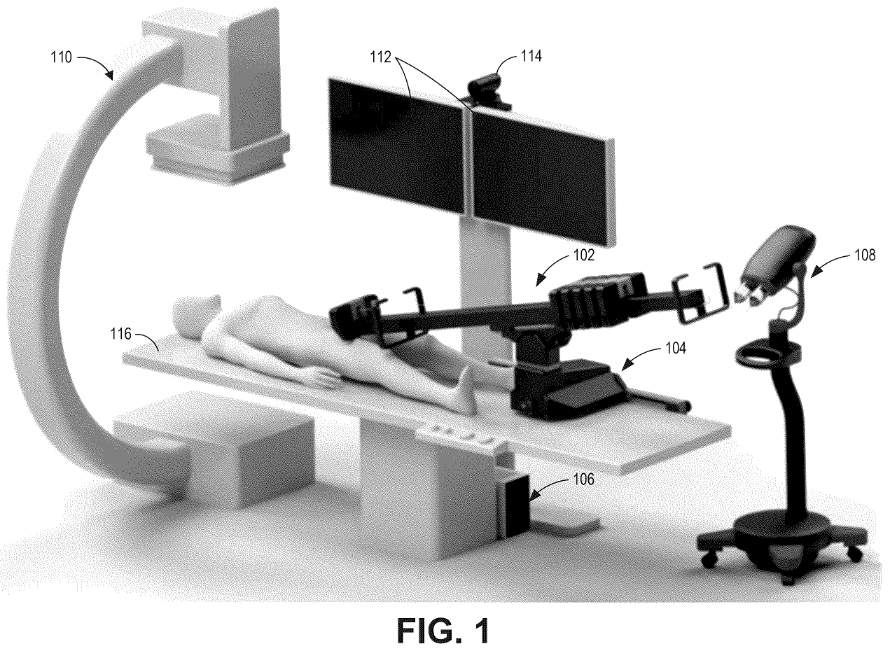

FIG. 1 illustrates a perspective view of an embodiment of some components of a robotic medical system 100. The robotic medical system 100 need not include each and every component shown in FIG. 1, and the robotic medical system 100 may also include other components not illustrated.

The robotic medical system 100 is configured to facilitate a medical procedure on a patient. In some embodiments, the medical procedure is an endovascular procedure. The robotic medical system 100 can include various components including an endovascular robot 102, a bed mount 104, a robot control unit 106, a contrast injector 108, a medical imager 110, one or more displays 112, a camera 114, and a bed 116. The robotic medical system 10 is configured to facilitate performance of an endovascular procedure on a patient positioned on the bed 116. Although not illustrated in FIG. 1, in some embodiments, the robotic medical system 100 may include medical drapes configured to maintain certain components sterile during a procedure.

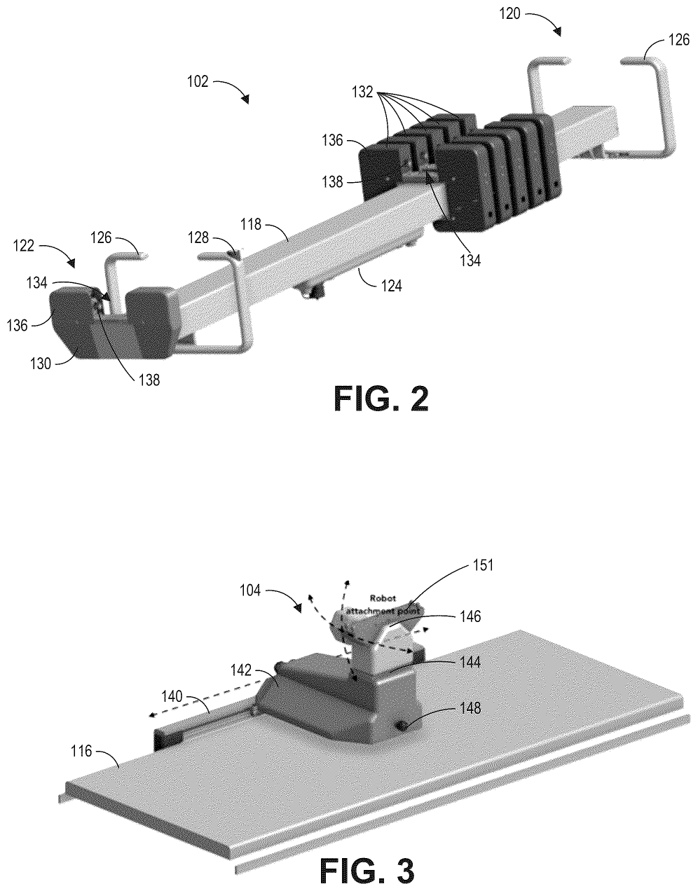

The endovascular robot 102 is shown in greater detail in FIG. 2, which illustrates another perspective view of the endovascular robot 102, shown in an undraped and unmounted position (e.g., not attached to the bed mount 104), illustrated with no tools attached thereto. In some embodiments, for example, as illustrated, the endovascular robot 102 comprises a generally long and slender device. In some embodiments, the endovascular device may comprise a length of about, at least, or at most 150 cm in length, although other lengths both longer and shorter are possible. In some embodiments, the endovascular robot 102 is configured to be lightweight, weighing, for example, less than 40 kg, less than 30 kg, less than 25 kg, or less than 20 kg. Reducing or limiting the weight of the endovascular robot 102 can facilitate installation and movement thereof.

In the illustrated embodiment of FIG. 2, the endovascular robot 102 comprises a rail 118. The rail 118 extends between a proximal end 120 (positioned away from the patient during use) and a distal end 122 (positioned towards the patient during use). The rail 118 can include an attachment device 124 that is configured to secure the endovascular robot 102 to the bed mount 104.

As illustrated in FIG. 2, in some embodiments, the endovascular robot 102 may include one or more handles 126. In the illustrated embodiment, the endovascular robot 102 comprises two handles 126, a first handle 126 positioned at the proximal end 120 of the rail 118, and a second handle 126 positioned at the distal end 122 of the rail 118. The handles 126 can provide locations at which a user (e.g., a medical professional in the operating environment) can grasp the endovascular robot 102 to, for example, reposition it relative to the patient. In some embodiments, during use, the endovascular robot 102 is secured to the bed mount 104 (e.g., see the illustrated configuration of FIG. 1) and the bed mount 104 is configured to provide one or more degrees of freedom that allow the endovascular robot 102 to be repositioned relative to the patient. The bed mount 104 is described in more detail below with reference to FIG. 3. The endovascular robot 102 can include a release button 128, which can be pressed to unlock the endovascular robot 102 and allow it to be repositioned. For example, in some embodiments, the position of the endovascular robot 102 is fixed until the release button 128 is pressed, and while the release button 128 is held, a user can reposition the endovascular robot 102. When the release button 128 is released, the position of the endovascular robot 102 can again be fixed. In the illustrated embodiment, the release button 128 is positioned on the distal handle 126, although other positions for the release button 128 are possible. For example, additionally or alternatively, the release button 128 can be positioned on the proximal handle 126.

In the illustrated embodiment of FIG. 2, the endovascular robot 102 includes a stationary helm 130 and a plurality of movable helms 132 positioned on the rail 118. In the illustrated embodiment, the stationary helm 130 is positioned at the distal end 122 of the rail 118. During use, the endovascular robot 102 is positioned such that the stationary helm 130 is positioned in close proximity to the endovascular access point on the patient (e.g., the patient's groin). The plurality of moveable helms 132 are positioned on the rail 118 and are configured to be independently moveable in proximal and distal directions, along the length of the rail 118. Various mechanical structures can be provided for independently moveable helms 132 along the length of the rail 118. For example, rack and pinion (where a pinion or circular gear within each moveable helm 132 is engaged with a rack or linear gear positioned within the rail 118), screw drives (where a nut within each moveable helm 130 is engaged with a screw positioned within the helm), belt and pulley, and or chain drive systems, among others, can be used for linear movement of the moveable helms 132 along the rail 118.

The stationary helm 130 and each of the plurality of moveable helms 132 can include an attachment location or recess 134 that is configured to receive an endovascular tool therein. The endovascular tools can be removably attachable to the recesses 134 of the stationary helm 130 and moveable helms 132.

Each of the moveable helms 132 can, in some embodiments, include some or all of the following features. The stationary helm 130 can also include some of these features. For example, each helm can include a housing 136. Each helm can include two articulation motors positioned within the housing (not visible in FIG. 2). Each of the articulation motors can be configured to drive a corresponding output 138, which can be configured as an output gear. When a disposable tool is loaded into the recess 134 of the helm, an input on the tool can engage with the output 138 such that the input of the tool can be driven by the articulation motor. Each of the moveable helms 132, can also include an insertion motor positioned within the housing 136 (not visible in FIG. 2). The insertion motor can be configured to cause movement of the moveable helm 132 along the rail. Because the stationary helm 130 is fixed in position, the stationary helm 130 need not include an insertion motor.

In some embodiments, each helm can include a torque sensor (not visible) associated with each motor (e.g., with each articulation motor and with the insertion motor). The torque sensor can be configured to calculate a torque associated with the motor. The torque sensors can be used to measure the force with which the tools are inserted into the patient and actuated. Signals from the torque sensor can be used to, for example, prevent tool insertion beyond a certain force threshold, stop the tool when the insertion force rapidly changes, ensure that the tools are fed into the patient at the same rate as the proximal end of the tool is advanced (in other words, eliminating slack between the feeder and tool), and/or enable precise control of the catheter pull wires associated with the tool.

Each helm can also include an RFID (radio frequency identification) tag reader. In some embodiments, tools that are attachable to the helms can be tracked through an embedded, sterile RFID tag. When a tool is loaded onto a helm, the helm can report the type of tool, as well as other characteristics such as expiration date, manufacturing lot number, tool length, and device-specific calibrations. This tracking ability allows the system to verify that everything is loaded correctly before starting the procedure.

Each helm can also include various electronic components positioned within the housing 136 (e.g., motor controllers, encoders, etc.).

As noted previously and as shown in FIG. 1, the endovascular robot 102 can be mounted to the bed 116 by a bed mount 104. An example embodiment of the bed mount 104 is shown in greater detail in FIG. 3. In the illustrated embodiment, the bed mount 104 is configured to mount to a rail 140 that is positioned on and/or attached to the bed 116. In some embodiments, for example, as illustrated, the rail 140 is positioned on a non-operator side of the bed 116. The bed mount 104 can include a base 142 that is attached to and configured to move linearly along the rail 140. A motor positioned with the base 142 can be configured to drive movement. Such movement can allow linear positioning of the endovascular robot 102 along the bed 116.

The base 142 of the bed mount 104 can include one or more joints (e.g., pivot or swivel joints) that provide additional degrees of freedom for positioning of the endovascular robot. For example, in the illustrated embodiment, the base 142 includes a yaw joint 144 and a pitch joint 146, to allow the yaw and the pitch of the endovascular robot 102 to be adjusted. The bed mount 104 illustrated in FIG. 3, thus allows for three degrees of freedom of adjustment for the endovascular robot 102: linear positioning along the rail 140, and yaw and pitch of the endovascular robot 102. These degrees of freedom are illustrated with dashed line arrows in FIG. 3. In other embodiments, other degrees of freedom can be provided by the bed mount 104 and/or one or more of the illustrated degrees of freedom can be omitted.

In some embodiments, during use, the bed mount 104 is configured so that the endovascular robot 102 can be positioned over the patient's legs (e.g., as shown in FIG. 1). Thus, the bed mount 104 can allow the distal end 122 of the endovascular robot 102 to be positioned in close proximity to an access point (e.g., a femoral sheath) positioned at the patient's groin.

In some embodiments, the bed mount 104 is configured such that, by default, the bed mount 104 is securely locked (e.g., the position is fixed). However, the position can be adjusted by pressing down on the release button 128 described above. That is, in some embodiments, the release button 128 can be held down in order to allow the position endovascular robot 102 to be adjusted. In some embodiments, the bed mount 104 includes one or more spring dampers that ensure that when the bed mount 104 is unlocked, the position of the endovascular robot 102 moves slowly and in a controlled manner to the desired position. Once the robot is correctly positioned, the operator can release the release button 128, and the bed mount 104 will automatically lock.

The bed mount 104 also includes a robot attachment point 151 that is configured to secure to the attachment device 124 on the endovascular robot 102 to secure the endovascular robot 102 to the bed mount 104.

The bed mount 104 can also include an emergency stop switch 148 that can be activated to stop all movement of the bed mount 104, the endovascular robot 102, and/or any tools attached thereto. As shown in FIG. 3, in some embodiments, the emergency stop switch 148 can be located on the front of the bed mount 104 to provide easy and unrestricted access to a bedside operator.

Additional detail regarding bed mounts that can be used with the robotic medical system 100 are described in U.S. application Ser. No. 19/049,670, filed Feb. 10, 2025, which is incorporated herein by reference.

For the robotic medical system 100, various tools can be configured to couple to the helms (e.g., the stationary helm 130 and the moveable helms 132). These tools can be configured to removably attach to the recesses 134 of the helms. In some embodiments, the tools are disposable, such that that are useable for a single procedure and the disposed of. Various tools can be used, including for example, one or more tools configured to linearly advance or retract an elongate device (e.g., a catheter or a guidewire), various catheters (both steerable and non-steerable, various guidewires, and the like. In some embodiments, the tools can include an RFID tag allows the helm to recognize which tool has been loaded onto the helm and whether or not it has been correctly loaded.

In some embodiments, endovascular tools (such as catheters) of the robotic medical system 100 can be configured to include one or more radio-opaque fiducials positioned thereon that are configured to be used in combination with computer vision to infer the pose of the catheter. In some instances, the term “pose” is used herein to refer to the position and orientation of a catheter. In some embodiments, determination of pose can be made based on a two-dimensional medical image, such as a single plane X-ray image, and one or more radio-opaque markers included on a catheter. Computer vision models can be employed to detect the catheter and/or radio-opaque markers positioned thereon in the two-dimensional medical image and to determine the pose of the catheter therefrom. In some instances, the pose can be defined by five degrees of freedom for the catheter. The five degrees of freedom can include two positional degrees of freedom (e.g., x and y position) and three degrees of freedom relating to orientation (e.g., heading, incline, and roll).

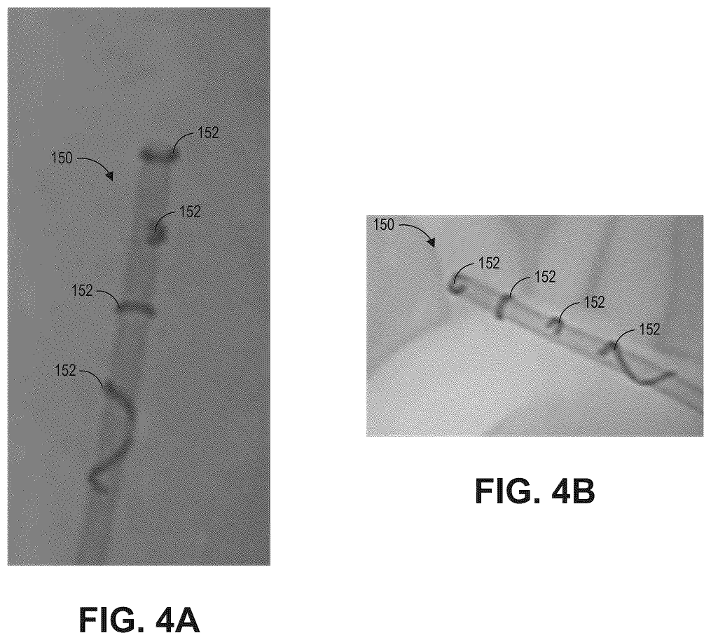

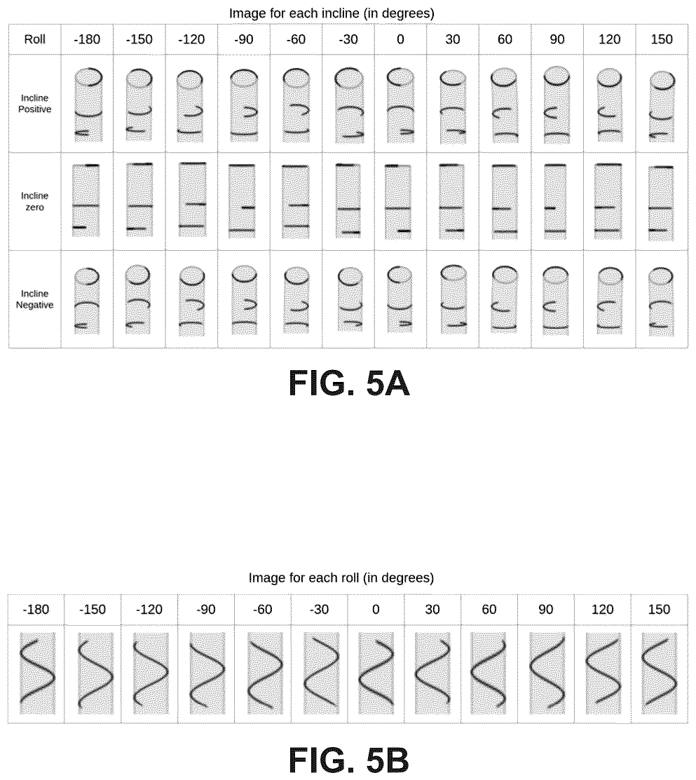

FIGS. 4A-5B illustrate how incline and roll of the catheter can be determined based on the two-dimensional appearance of the radio-opaque markers included on the catheter. Roll is measured around a longitudinal axis of the catheter. Incline is measured relative to the plane of the medical image (e.g., whether the catheter is inclined into or out of the plane of the medical image). FIGS. 4A and 4B, for example, are fluoroscopic images of a catheter 150 configured with radio-opaque fiducials 152 that allow determination of catheter pose using a computer vision algorithm. In the illustrated embodiment, the radio-opaque fiducials 152 comprise three the non-circumferential rings that are semi-circular, extending part way around the catheter 150. In some embodiments, the non-circumferential rings 152 can be radio-opaque such that it can easily be identifiable within a medical image. The appearance of the non-circumferential rings 152 in a two-dimensional medical image can be analyzed, using computer vision to determine the sign and magnitude of the incline of the catheter 150. The sign and magnitude of the incline of catheter 150 can be determined by the unique appearance of the non-circumferential rings 152 in the two-dimensional image at varying degrees of incline, both positive and negative. In some embodiments, non-circumferential rings 152 are arranged in an asymmetrical design. That is, in some embodiments, the non-circumferential rings 152 are each positioned at a different rotational position around the catheter 150. In the illustrated embodiments, the non-circumferential rings are positioned at 90-degree offsets. In some embodiments, non-circumferential rings 152 are multiple ellipses offset from each other.

FIG. 5A illustrates how the example arrangement of non-circumferential rings 152 shown in FIGS. 4A and 4B positioned on the distal end of the catheter 152 may provide a unique appearance at different inclination and roll angles. Images are provided at positive, neutral (i.e., zero), and negative inclinations, as well as at different roll positions provided in 30-degree increments. As shown, each of the 36 different illustrated positions provides a unique appearance. By detecting, for example, using computer vision, this appearance within a medical image, the incline (including its sign) and the roll of the catheter can be determined. While FIG. 5A illustrates how the radio-opaque markers 152 provide different two-dimensional appearances for different roll positions at 30-degree increments and for different positive, neutral (zero), and negative inclines, the illustrated increments are not intended to be limiting.

Returning to FIGS. 4A and 4B, the illustrated embodiment of the catheter 150 also includes a different radio-opaque fiducial 152 having a helical shape. FIG. 5B illustrates the two-dimensional appearance (e.g., as within the plane of medial image) of the helical fiducial 152 of FIGS. 4A and 4B and different roll positions in 30-degree increments. As shown, each roll position provides a unique appearance which can be used to determine roll, for example, by a computer vision, neural network, or machine learning system. While FIG. 5B illustrates how the radio-opaque markers provide different two-dimensional appearances for different roll positions at 30-degree increments, the illustrated increments are not intended to be limiting.

In some embodiments, a computer system may utilize artificial intelligence or machine learning to perform such functionality. In some embodiments, for example, a neural network can be trained to detect the position/appearance of the radio-opaque fiducials in combination within a two-dimensional image, and extract or determine the incline and/or roll from the detected appearance position. It should be noted that in some embodiments, the machine learning algorithm does not hardcode the aforementioned approach. Instead, the machine learning algorithm trains a deep neural network to directly predict the incline angle or roll from the input of the X-ray image.

Other methods and mechanisms can be used for determining the roll and incline of a catheter in the robotic medical system 100. Further, for embodiments that include radio-opaque fiducials, other configurations for the radio-opaque fiducials can be used.

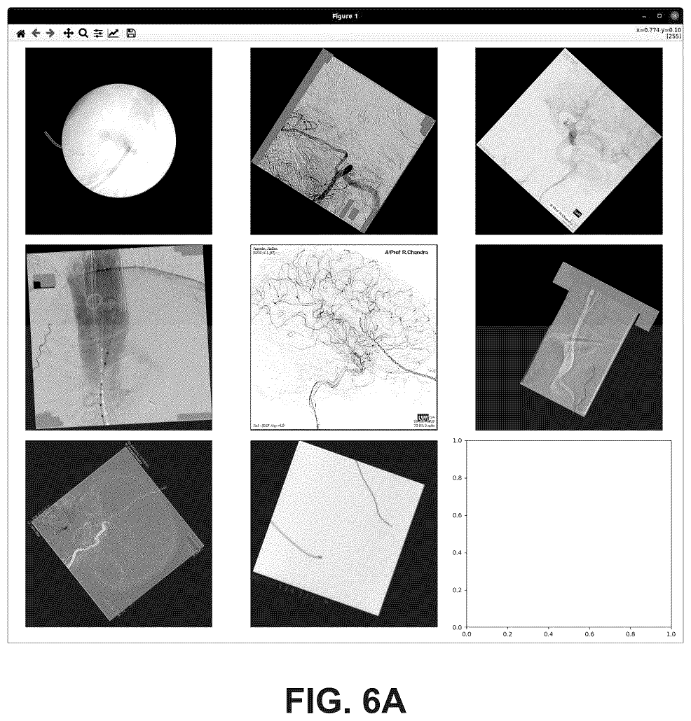

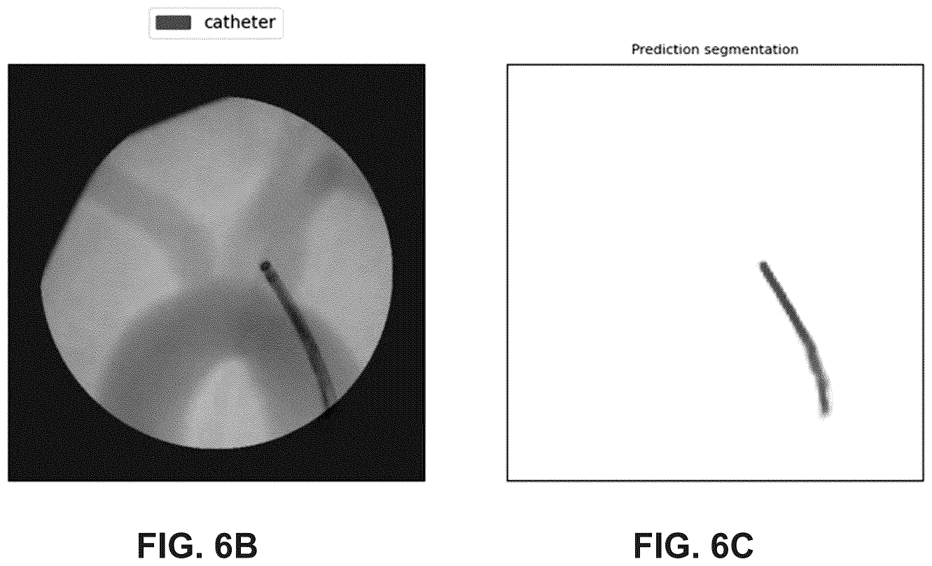

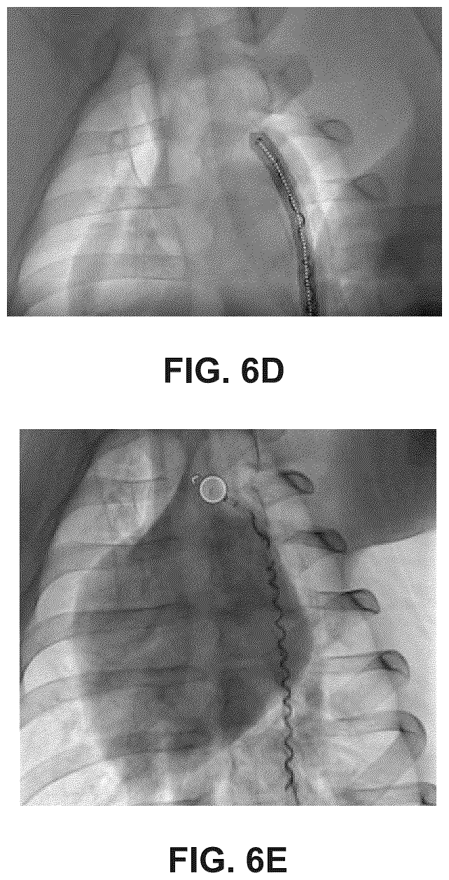

Machine learning and/or computer vision can also be used to determine position (e.g., x, y position) and heading (e.g., direction within the plane of the medical image) of a catheter. FIG. 6A illustrates examples of projections of three-dimensional generated catheters onto real world two-dimensional X-ray images. FIGS. 6B-6C illustrate an example prediction of the trained deep neural network for predicting where the catheter body is. As noted above, position can refer to translation of endovascular and/or other intraluminal tools along the x, y directions in the plane of the medical image. In some embodiments, the system may be configured to predict where the full tool body is, and then from this tool body, the two-dimensional tip location can be extracted. This approach may be beneficial because the tool body provides a very strong training signal for learning deep neural network segmentation models. That is, in some instances, it may be easier for a neural network of computer vision algorithm to detect the body of a catheter and then extract the location of the tip from there. In some embodiments, catheter kinematics are further used to refine this estimate.

For example, a deep neural network can be used to estimate the two-dimensional centerline position of the catheter based on one or more images of the catheter navigating within the body. FIG. 6D illustrates an example, in which the neural network has identified the catheter within the image, superimposed its estimated centerline onto the image, and highlighted the catheter. Once the centerline of the catheter has been identified within the image, the two-dimensional position can be directly extracted by computing the most distal position along the centerline. Similarly, the heading of the catheter can also be directly extracted from the body estimate by computing the vector of the tip of the body line. FIG. 6E illustrates an example in which the distal tip position and heading angle have been determined and the image has been updated to include a highlight identifying the position and an arrow indicating the heading.

In some embodiments, a machine learning algorithm for estimating the position of a catheter and/or other tool may use the following approach. First, the image generation procedure is modified by drawing the catheter on top of the medical image (e.g., as shown in FIG. 6B). This process may have the advantage of training the deep neural network with realistic noise and occlusions that would be seen in actual X-rays, making the system robust to real world conditions.

Second, the two-dimensional x and y position is estimated. In some embodiments, radio-opaque markings may be added to the tool body, such as, for example, a full-length helix, to assist with the identification. Thereafter, the two-dimensional x and y position location of the tool tip can be determined. This approach may be used because the tool body provides a very strong training signal for learning deep neural network segmentation models. In some embodiments, catheter kinematics may be used to further refine the position estimate.

To determine the heading of endovascular and/or other intraluminal tools, such as a catheter, the deep neural network prediction of the catheter body position may be used. Based on the prediction of the two-dimensional x and y position of the catheter tip, a second position located on the catheter body may be determined. The second position may be an infinitesimal distance from the tool tip in a direction along the catheter body. The heading angle of the catheter may then be calculated using trigonometry based on the x and y position of the tool tip and the second position along the catheter body.

Additional detail regarding pose determination is described in U.S. application Ser. No. 17/819,101, filed Aug. 11, 2022, and granted as U.S. Pat. No. 11,707,332 on Jul. 25, 2023, each of which is incorporated by reference herein in its entirety. Other methods and mechanisms for determining position and heading of the catheter may also be used.

Thus, in some embodiments of the robotic medical system 100, computer vision can be used to infer tool position, orientation, incline and roll, we solve this problem and make catheter control simple. That is, computer vision can analyze a medical image to determine the tool position (e.g., as represented by a two-dimensional line corresponding to the centerline of the catheter), tool tip position (e.g., as represented as an x, y coordinate in the plane of the medical image); tool tip heading (e.g., the direction the catheter is pointing in the image plane), tool incline (e.g., to the degree to which the tip is pointing up towards the x-ray imager or down away from the x-ray imager), and/or tool roll (e.g., how the catheter is rotated about itself).

By understanding all of these position components of the tool, the robotic medical system can be configured to control the catheter autonomously or, at the very least, make control for the user extremely simple.

For example, in some embodiments, the robotic medical system 100 can be configured to allow for “image space control” wherein control inputs are provided with respect to how the tool appears in the medical image, regardless of the current orientation of the tool. This type of control system is intuitive as the user may provide such inputs while viewing the medical image which includes at least a representation of a distal portion of the instrument. That is, the user can provide control inputs relative to the current appearance of the instrument within a medical image. In this way, the user can provide natural and intuitive inputs with respect to the current position and orientation of the instrument within a medical image, and the system can determine appropriate motor commands (e.g., commands for actuating one or more of the pullwires of the instrument) to cause the desired motion. In some embodiments, this can allow the user to control the catheter in one or more of the following three directions: forward and back (insertion), left and right (heading), and/or into and out of the image (incline). These directions move with respect to the plane of the image regardless of how the X-ray is moved or how the catheter is rolled in the body. This control mode is intuitive and provides a large advancement over the current standard of care, which requires the user to frequently guess and check which way the catheter will move on screen. Using these controls, the user can easily access tricky vessels and ensure safe navigation of the instrument through the vessels in an atraumatic fashion.

A user may provide user inputs in various ways. For example, in some embodiments, the user can specify desired targets for insertion, heading, and/or incline. Once specified, the system can determine the appropriate motor commands for causing the instrument to move from its current position and orientation to the desired position and orientation. Providing such absolute targets (e.g., desired targets for insertion, heading, and/or incline) may advantageously provide some resiliency and safety in the event in a lag in communication between the user and the robotic medical system. This can be advantageous for situations wherein the user is remotely located from the robotic system and patient and communication occurs of a computer network, such as the internet.

As another example, a user may provide user inputs that are indicated relative to the current position or orientation of the instrument. For example, a user can specify that the instrument adjust its heading to the right relative to the current heading of the instrument, While such a system may be less tolerant to high latency and communication lag, it still allows user to navigate in a simple and intuitive manner.

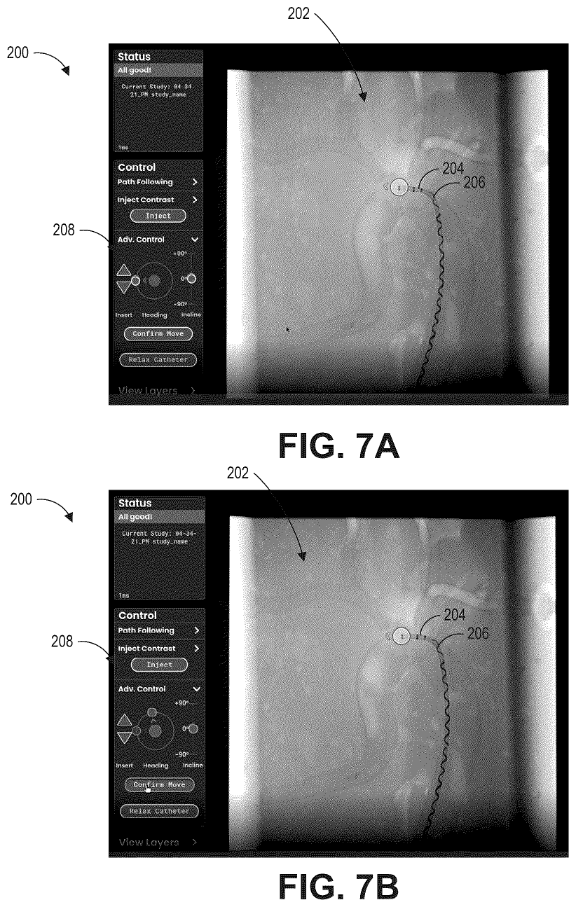

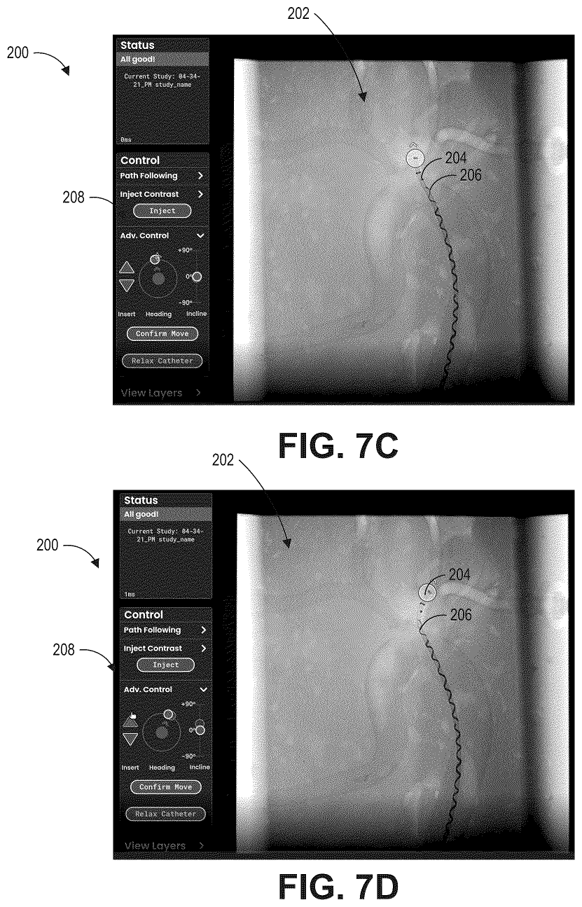

FIGS. 7A-7D illustrate an embodiment of a graphical user interface 200 for providing image space control of a medical instrument. In the illustrated embodiment, the graphical user interface is configured to display a two-dimensional medical image 202, such as an X-ray. The medical image 202 includes a view of a distal end of a medical instrument, such as a catheter 204. The catheter 204 includes one or more fiducials 206 positioned thereon. The fiducials 206 are visible within the medical image 202. The fiducials 206 can be configured as described above in order to allow for vision-based determination of the position and orientation (including roll) of the medical instrument. For example, at least one fiducial 206 can be configured such that it provides unique two-dimensional appearances associated with different roll angles for the catheter 204, for example, as described above with reference to FIGS. 4A-5B.

The graphical user interface 200 may also include a user input device 208. The user input device 208 is configured to receive user inputs from a user that are provided with respect to the two-dimensional medical image 202. For example, in the illustrated embodiment, the user input device 208 includes features for allowing a user to input insert commands (e.g., to advance or retract the instrument 204), heading commands (e.g., to alter the heading of the medical instrument 204 within the plane of the medical image 202, for example, to the right or left of the instrument's current heading), and incline commands (e.g., to alter the incline of the medical instrument 204 into or out of the plane of the medical image 202. The user input device 208 may include other options as well. For example, in the illustrated embodiment, the user input device 208 includes options to inject contrast, confirm an entered movement, and to relax the catheter.

Although the user input device 208 is illustrated as a component of the graphical user interface 200, this need not be the case in all embodiments. For example, in some embodiments, the user input device 208 can comprise a handheld control.

Importantly, the user input device 208 allows the user to provide user inputs for controlling the instrument 204 with respect to the current configuration of instrument as shown in the two-dimensional medical image 202. For example, as shown in FIG. 7B, the user may input a desired heading for the medical instrument via the heading input of the user input device. In the illustrated configuration, the user can input a desired heading by selecting a target point on the wheel. In FIG. 7B, the desired heading is shown at about 355 degrees with a highlighted circle. The current heading is also shown on the wheel at about 270 degrees as a lighter circle. The user may also select a desired inclination using the incline slider, if desired.

Continuing this example, with reference to FIG. 7C, by selecting the confirm move option, the robotic system can determine appropriate motor commands to cause the instrument 204 to move to the desired heading and incline. FIG. 7C shows the instrument 204 after movement. FIG. 7D illustrates that, by using the insert arrows of the user input device 208 the user can command forward and backward motion of the instrument 204.

The graphical user interface 200 and user input device 208 of FIGS. 7A-7D provide only one example of how these features may be configured.

In order to generate appropriate motor commands based on the user inputs to cause the instrument to move appropriately, it is important that the current roll of the instrument be accounted for. This is necessary to ensure that the appropriate pullwires are actuated to cause the specified motion. In some embodiments, the system determines the roll of the instrument automatically, for example, using computer vision analysis of the appearance of one or more of the fiducials in the image as discussed above.

Returning to FIG. 1, the robotic medical system 100 may also include a robot control unit 106, a contrast injector 108, a medical imager 110, one or more displays 112, and a camera 114.

The robot control unit 106 may be a computerized device including a processor and computer memory storing instructions that configure the process to facilitate control of the robotic medical system. For example, the robot control unit 106 unit may facilitate determination of the pose of a medical instrument by analyzing a medical image provided by the medical imager 110. Similarly, the robot control unit 106 may facilitate image space control by translating image space control user inputs into appropriate motor commands for moving a medical instrument.

The contrast injector 108 can be configured to supply contrast to the robotic medical system such that contrast can be injected to facilitate visualization of the vasculature. In some embodiments, the contrast injector 108 comprises a dual headed system, where one head is loaded with radiopaque contrast and the other head is loaded with saline. In other embodiments, the contrast injector can comprise a single headed system, with one head loaded with radiopaque contrast and a heparinized saline flush is hung. The lines can be connected and drain into a single common line which is attached to a connector on the back of a steerable catheter before being flushed and checked that they are free of air.

The medical imager 110 can comprise an X-ray device, such as a C-arm. The medical imager 110 is configured to capture medical images (e.g., X-rays) of the patient during a medical procedure.

Displays 112 can be provided in the operating environment for visualization by medical personnel located onsite. A camera 114 can be positioned to capture an image of the operating environment to, for example, allow a remote operator to visualize the procedure.

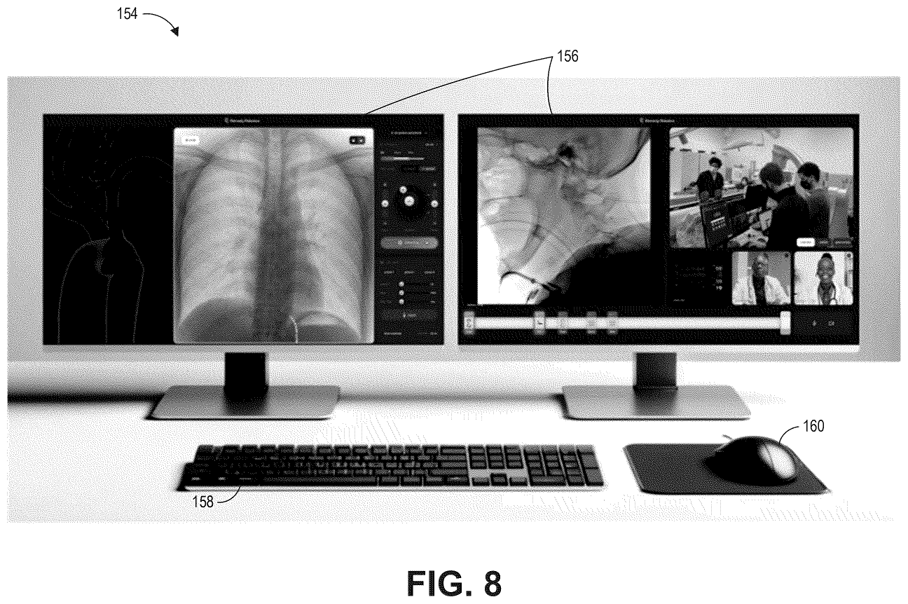

The robotic medical system 100 can be configured to be controllable using computerized equipment, such as a personal computer, a laptop, or a tablet. An example user control station 154 is shown in FIG. 8. This user control station 154 is provided only by way of example, and the user control station 154 may be provided in other forms as well.

In the illustrated embodiment, the user control station 154 comprises a personal computer (not shown), two displays 156, a camera and microphone (not shown), a keyboard 158, and a mouse 160. In some embodiments, the user control station 154 may also comprise a foot pedal (not shown).

In the illustrated embodiment, the user control station 154 is configured to display a graphical user interface. In the illustrated embodiment, left display 156 is configured to display a three-dimensional model of the vasculature of the patient, which can be rotated and manipulated by the user. The left display 156 is also configured to display an X-ray feed, showing an image from the medical imager 110. In the illustrated example, the x-ray feed occupies the majority of screen space on the left display 156. A “live” icon indicates when the X-ray image displayed is a live image. In some embodiments, a co-registered model of the vessels can be overlaid on the X-ray image. The co-registered model can be toggled on and off. The left display 156 can also include user controls for selecting which tools to control, injecting contrast, and moving the tools. In some embodiments, the left display 156 can include image space control user inputs.

In the illustrated embodiment, the right display 156 is configured to display an X-ray feed for biplane imaging, a toggleable camera views directly into the Cath lab (e.g., a view from camera 114), displays of remote viewers and hemodynamics, and a schematic view of the endovascular robot 102 to show which tools are active and show where the helms are in reference to one another.

The illustrated graphical user interface is provided as only one example. Other embodiments can include some, none, or other features. Further, the features on the graphical user interface can be arranged differently. In some embodiments, the graphical user interface can be displayed on a single display so as to be useable on a tablet, smartphone, or laptop. In some embodiments, the user control station 154 can be configured for touchscreen inputs.

Notably, the user control station 154 can be remotely located from the remainder of the robotic medical system 100 components shown in FIG. 1 and can communicate over a network, including the internet. In this way, a remotely located operator can control the robotic medical system 100.

The robotic medical system 100 can be configured to enable a local or remote operator to navigate catheters from the femoral or radial artery or vein to the proximal cerebral, distal cerebral, coronary, pulmonary, aorta, peripheral arteries or veins as well as inject contrast and aspirate a thrombus, deliver a stent retriever, deploy a coil, deploy a stent, and/or deliver a liquid embolic, among other features and procedures.

### Background Task and Safety Enable Input

In some embodiments, a robotic medical system, such as the robotic medical system 100 described above, can include a background task and safety enable input (referred to herein simply as an “enable input”). The enable input can comprise a user input that can be provided, for example, as part of the user control station 154 (e.g., as described above with reference to FIG. 8). That is the enable input can be a user input that an operator of the robotic medical system can provide into the system. In some embodiments, the enable input is provided by way of a foot switch, such that the user can provide the enable input without requiring the use of either hand. In other embodiments, the enable user input can be provided via keyboard, mouse, touchscreen, or any other control or input mechanism.

The enable input can be an input that must be activated in order for the robotic medical system 100 to take any action. For example, when the enable input is not activated, the robotic medical system 100 can be prohibited from taking any action. In this way, the enable input can serve as a safety in that the robotic medical system 100 can only perform an action when the enable input is actuated. For example, when the enable input is a foot switch, the robotic medical system 100 can only perform an action when the foot switch is pressed. For example, a user may be required to press and hold the foot switch in the activated position to signal the robotic medical system 100 that it can perform an action. If the foot switch is not depressed, no action is taken. This can prevent an inadvertent command from being sent to the robotic medical system 100 which can prevent unintended movement of the endovascular robot 102 or any tools coupled thereto. This can facilitate patient safety. For example, if a user accidentally or unintentionally provides a user input to advance a catheter coupled to the endovascular robot 102, such command would not cause motion unless the enable input is also actuated. That is, commanded motions are only performed when the enable input is actuated at the same time that the commanded motion is provided.

Additionally, the enable button can also be used as a signal to the robotic medical system 100 that it can perform various background tasks. In this way, the enable button can be considered an “explicit” command to perform automated functions, which would otherwise run in the background without providing any user input. This may be advantageous as certain regulatory bodies may require that explicit user commands be provided in order for the robotic medical system 100 to perform any action. This may also improve safety as the robotic medical system 100 will only perform automated or background tasks when the user is providing the enable input, which will likely occur only when the user is carefully monitoring the system. In general, background tasks are performed when the enable input is activated but no motion commands are provided by the user. This advantageously allows the robotic medical system to perform automated background tasks in between commanded motions, as long as the enable input is actuated.

As used herein, a motion command refers to any explicit command provided by the user to move the endovascular robot 102, e.g., motions that cause insertion or retraction of a catheter, wire or other tool, motions that cause roll of a catheter, wire, or other tool, motions that cause articulation or deflection of a steerable catheter, motions that actuate a tool (e.g., cause deployment of a stent, cause aspiration, etc.), among others. As described above, the enable input can be required to be actuated (e.g., depressed or held) in order for the robotic medical system to act on a motion command. In some embodiments, the robotic medical system 100 may require the enable input to be actuated in order to perform other tasks that may require user input such as causing delivery of contrast via the contrast injector 108 or activating the medical imager 110.

As noted previously, when the enable input is actuated but no other user inputs are provided, the robotic medical system 100 may perform background or automated tasks. As used herein, background tasks may comprise tasks that are performed by the robotic medical system 100 including the tasks performed by the endovascular robot 102 that do not result in any motion at the tip of the tools inserted into the patient's vasculature. As one example, a background task can include taking steps to reduce or minimizing slack or buckling of any tools. Slack or buckling management is described in more detail in the following section. The features for managing slack and buckling described in the following section can be performed automatically or in the background while the enable input is actuated and no other explicit user commands or motion commands are provided. As another example, a background task can include resetting a position of a moveable helm 132 or tool attached thereto in a manner that does not result in motion at the tip of the inserted tools. For example, a tool coupled to a moveable helm 132 can disengage from a catheter or wire passing there through and move over the catheter or wire to a new position before reengaging the tool. This can ensure that the moveable helms 132 are always in advantageous positions in preparation for future movements. For example, in the event that a moveable helm 132 is too close to a distal end 122 of the rail 118 or to another moveable helm 132, while the enable input is actuated but no motion command is provided, the moveable helm 132 can, in the background and in an automated manner, reset itself to a more advantageous position.

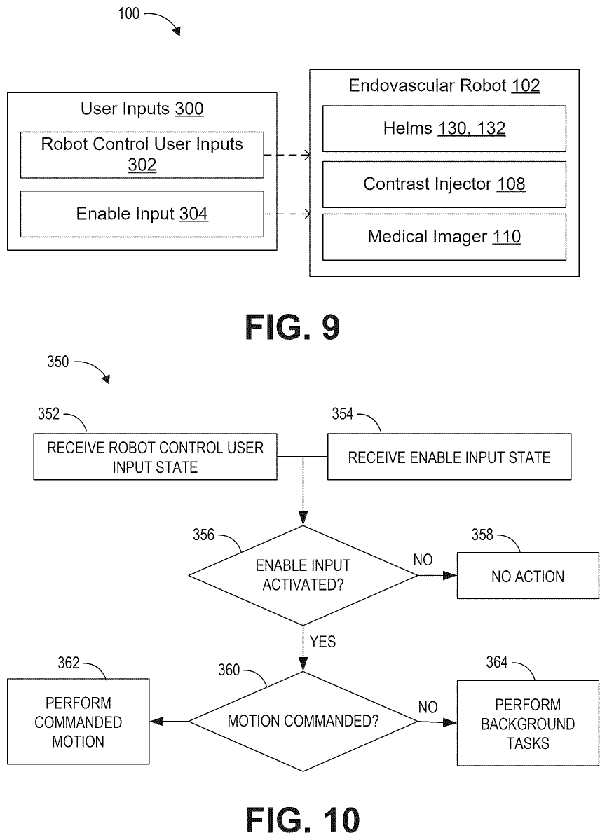

FIG. 9 illustrates a simplified schematic version of the robotic medical system 100, illustrating certain features relating to the enable input described in this section. As shown, the robotic medical system can include user inputs 300. The user inputs 300 can be provided as part of the user control station 154. In the illustrated example, the user inputs 300 comprise robot control user inputs 302 and an enable input 304. The robot control user inputs 302 can be user inputs that a user uses to provide explicit motion and other action comments to the robotic medical system 100. These can be, for example, user inputs indicative of causing insertion or retraction of a catheter, wire or other tool, causing roll of a catheter, wire, or other tool, causing articulation or deflection of a steerable catheter, causing actuation of a tool (e.g., cause deployment of a stent, cause aspiration, etc.), among others. These may also be, for example, user inputs for causing delivery of contrast via the contrast injector 108 or activating the medical imager 110. In some embodiments, the robot control user inputs 302 can be image space control user inputs.

The enable input 304 can be a user input that is actuated by the user to allow the robotic system to perform any action at all. For example, the enable input 304 can be required to be activated before the system acts on any commands provided by way of the robot control user inputs 302. As discussed previously, the enable input 304 can also provide a signal that, in the absence of any robot control user inputs 302, permits the robotic medical system 100 to perform background or automated tasks. The robotic medical system 100 can only perform background or automated tasks when the enable input 304 is actuated.

In some embodiments, the enable input 304 can be provided as a footswitch. In some embodiments the footswitch must be depressed and held to activate the enable input 304. In other embodiments, the enable input 304 can be provided via other buttons, keyboard selection, touchscreen inputs, mouse inputs, or other types of user inputs.

As shown in FIG. 9, outputs from the robotic control user inputs 302 and the enable input 304 are provided to the endovascular robot 102 (as illustrated with dashed arrows). While the enable input 304 is actuated, components of the endovascular robot 102, such as helms 130, 132, contrast injector 108, and/or medical imager 110 can perform actions based on the robot control user inputs 302. That is, if a robot control user input 302 is sent while the enable input 304 is not active, the robotic medical system will perform no action. If the enable input 304 is active, the robotic medical system will act on the robot control user inputs 302. If the enable input 304 is active and no robot control user inputs 302 are provided, the robotic medical system can perform background or automated tasks as described above.

FIG. 10 is a flow chart illustrating a method 350 for operating the robotic medical system 100 that includes a background task and safety enable input. At block 352, the method 350 receives a robot control user input state. This can involve checking to see whether a user is or has provided a robot control user input (e.g., a user input for the robotic medical system to take an action). At block 354, the method 350 receives an enable input state. This can involve checking to see whether the enable input is active. In some embodiments, blocks 352 and 354 can occur simultaneously or substantially simultaneously (e.g., within a time window (e.g., with 5 milliseconds, 10 milliseconds, 20 milliseconds, 30 milliseconds, 40 milliseconds, 50 milliseconds, 75 milliseconds, or 100 milliseconds, for example)).

The method 350 moves to decision state 356. At decision state 356, the method 350 checks whether the enable input state indicates that the enable input is active. If the enable input state is not active, the method 350 moves to block 358, which prohibits the robotic medical system from taking any action. If the enable input state is active, the method 350 moves to decision state 360. At decision state 360, the method 350 checks whether the robot control user input state is indicative of a commanded motion. If the robot control user input state is indicative of a commanded motion, the method 350 moves to block 362 and the commanded motion is performed. If the robot control user input state indicates that no motion is commanded, then the method 350 moves to block 364 at which the robotic medical system performs background or automated tasks.

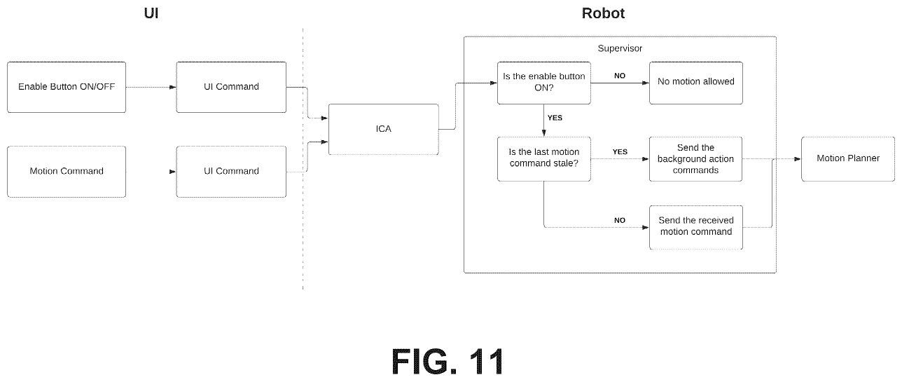

FIG. 11 illustrates a proposed architecture for facilitating a robotic medical system having a background task and safety enable input. As illustrated in FIG. 11, a user interface (UI) can send a periodic enable button status message, letting a supervisor control system (“supervisor) know whether the enable button is active or not. In this proposed architecture, the enable button status does not impact the ability to send other commands. In this proposed architecture, whether the motion commands should be acted upon will be handled within the supervisor.

The proposed architecture of FIG. 11 assumes that the enable button status message will be sent periodically (e.g., at a rate of 10 Hz) as long as the enable button is pressed. Once the enable button is released, a final command will be sent informing the supervisor that the enable button has been released. In this proposed architecture, while the enable button is released, no periodic message will be sent. Similarly, the UI can send motion commands to the supervisor.

As shown in FIG. 11, the enable button status message and motion commands are sent via UI commands to the supervisor. The supervisor will now receive two messages regarding motions: the actual motion command and the enable button status. These two messages come in independently from each other, and thus must be “synced” to ensure that the robotic medical system does not allow motions when the enable button is not pressed. The synchronization can, for example, be accomplished via an independent component analysis (ICA) module, which makes use of a duration value that estimates whether the system is no longer expecting a UI command, both for the enable button and for the explicit motion command. For example, the ICA module can, for the enable button, determine if a time period of milliseconds have passed since receiving an enable button status message, automatically assume the enable button is no longer pressed. Similarly, the ICA module can, for the motion command, determine that if a time period of milliseconds have passed since receiving the last motion command, automatically assume that no motion command is being requested.

As shown in FIG. 11, if the enable button is not on, no motion is allowed. If the enable button is on, and a last motion command is current, the motion command is sent to the motion planner and executed. Conversely, if the enable button is on and the last motion command is stale (e.g., no motion is commanded), background action commands are sent to the motion planner such that the system performs the background tasks.

### Slack and Buckling Control

The robotic medical system 100 can be configured with functionality to control (e.g., reduce or minimize slack and buckling of endovascular tools (e.g., catheter or wires)) inserted into the patient's vasculature. Minimizing slack and buckling of endovascular tools can facilitate more accurate control and movement of the endovascular tools and improve patient safety. As used herein, slack refers to bowing of sagging of an elongated body of an endovascular tool that occurs outside of the patient's body. As used herein, buckling occurs to unintended bowing of the elongated body of an endovascular tool that occurs inside of the patient's body. In some embodiments, slack and buckling control can be performed as a background task (e.g., automatically be the robotic medical system), for example when the enable input is activated but no motion commands are provided as described above. In other embodiments, slack and buckling control can be performed in response to a user command. For example, in some embodiments, a user may provide an explicit command to trigger the slack and buckling control functions described in this section.

As noted above, in this application, slack is used to refer to bowing or sagging of an elongated body of an endovascular tool (e.g., a catheter or wire) that occurs outside of the body. In many instances, slack can occur in a portion of an endovascular body that extends between the distal helm 130 and the nearest moveable helm 132 or between any two adjacent moveable helms 132. Slack can occur, for example, as the endovascular tool is inserted into the patient if the rate of insertion of the distally located helm 130, 132 is slower than the rate of insertion of a proximally located helm 132.

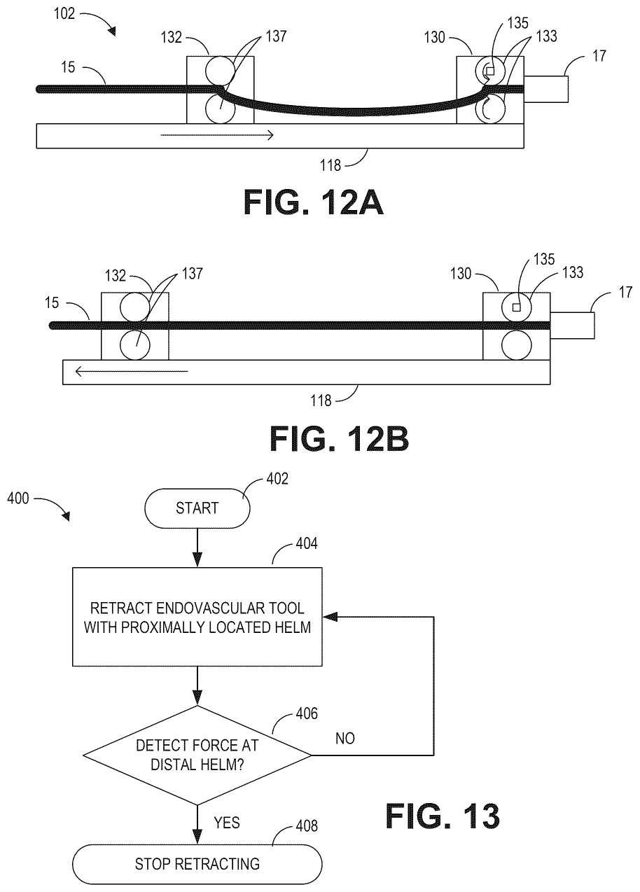

FIG. 12A illustrates an example showing slack of an endovascular tool 15 between the distal helm 130 and a moveable helm 132 located proximally to the distal helm 130. In this example, the distal helm 130 is fixed at the end of the rail 118 and is configured to cause insertion or retraction of the endovascular tool 15 via rollers 133 on the distal helm 130 (or on a disposable tool coupled to the distal helm 130. The rollers 133 contact the elongated body of the endovascular tool 15. When rotated in one direction (for example, in the direction indicated by the arrows) the rollers 133 cause insertion of the endovascular tool 15 into the patient through the introducer 17. Rotation of the rollers 133 in the opposite direction can cause retraction of the endovascular tool 15. The rollers 133 can be rotated by motors positioned within the distal helm 130. A force or torque sensor 135 can be associated with one or both of the rollers 133 and/or the motors that drive the rollers 133. Because the rollers 133 engage with and drive the endovascular tool 15 via friction between the rollers and the endovascular tool 15, it is possible that some slippage can occur. Thus, in some instances, the rate at which the endovascular tool 15 does not exactly match the rate at which the rollers 133 rotate.

As shown in FIG. 12A, the endovascular tool 15 also passes through a moveable helm 132. The moveable helm 132 can also be engaged with the endovascular tool 15. In the illustrated embodiment, the moveable helm 132 is engaged with the endovascular tool 15 via grippers 137 that pinch the body of the endovascular tool 15 to securely hold it. In some embodiments, the grippers 137 can be included on the moveable helm 132 or on a removable tool coupled to the moveable helm 132. The moveable helm 132 can be configured to advance the endovascular tool 15 by moving in the distal direction along the rail 118 (for example, in the direction indicated by the arrow). The moveable helm 132 can be configured to retract the endovascular tool 15 by moving in the proximal direction along the rail 118. In some embodiments, the moveable helm 132 (or tool removably coupled thereto) can include rollers similar to the distal helm 130. The rollers could be used to advance or retract the endovascular tool 15 through the moveable helm 132 without requiring movement of the moveable helm 15.

Ideally, the endovascular tool 15 would extend in a substantially taut manner between the distal helm 130 and the proximally located moveable helm 132. However, if the rate at which the distal helm 130 advances the endovascular tool 15 is slower than the rate at which the moveable helm advances the endovascular tool 15, the endovascular tool 15 will begin to droop or sag (e.g., slack) between the two helms 130, 132. Slack is undesirable for several reasons including that it leads to imprecise control of the endovascular tool 15 and too much slack can cause the endovascular tool 15 to come into contact with portions of the endovascular robot 102 that are not intended.

As described in this section, the robotic medical system 100 can be configured to manage (e.g., reduce or eliminate) slack. Slack can be managed by retracting the endovascular tool 15 with the proximally located moveable helm 132 while not retracting the endovascular tool 15 with the distal helm 130 until the force or torque sensor 135 receives a signal indicative of a spike in force or torque that exceeds a threshold. The threshold can be set at a torque or force that is indicative of the endovascular tool 15 between the two helms 130, 132 going taut.

An example is shown, in FIG. 12B, as shown, the proximally located moveable helm retracts in the proximal direction until the endovascular tool 15 between the two helms 130, 132 goes taut. This causes the force or torque sensor 135 associated with the distal helm to experience a signal spike. If this spike exceeds a preset threshold, the moveable helm 132 stops retracting the endovascular tool 15. As shown in FIG. 12B, the slack is thus eliminated.

FIG. 13 illustrates an example method 400 for removing slack in a robotic medical system. The method 400 can manage slack between a distally located helm and a proximally located helm. The distally located helm can be the stationary helm 130. In other embodiments, the distally located helm can be a moveable helm 132, and slack can be managed between two moveable helms 132.

The method 400 begins at block 402. In some embodiments, block 402 is triggered based on a user input. For example, a user may provide an input to begin the slack removal process. In other embodiments, slack may be managed automatically, and the robotic medical system may automatically begin the slack removal process when no movement commands are provided by the user. The slack removal process can be performed automatically as a background task when the enable input described previously is active, but no motion commands are provided.

Once the method 400 begins, at block 404, the endovascular tool can begin to be retracted using the proximally located helm. At the same time, the distally located helm remains stationary (e.g., does not retract the endovascular tool). The method 400 moves to decision state 406 while the proximally located helm continues to retract the endovascular tool. At decision state 406, the system evaluates whether a sufficient force or torque is detected using the force or torque sensor on the distally located helm. If no sufficient torque or force is detected, the method 400 returns to block 404 and continues to retract the endovascular tool with the proximally located helm. If, at decision state 406, a sufficient force or torque is detected at the distally located helm, the method 400 moves to block 408 and stops retracting the endovascular tool with the proximally located helm. The torque or force detected at decision state 406 can be a force or torque which exceeds a predetermined threshold indicative of the endovascular tool going taut between the two helms indicating that slack has been removed.

As noted above, in this application, buckling is used to refer to unintended bowing of an elongated body of an endovascular tool (e.g., a catheter or wire) that occurs inside of the body of the body. In many instances, buckling can occur when an endovascular tool is advanced into the body especially when a proximal portion of the endovascular tool advances a distance that is greater than the distal tip of the endovascular tool moves forward. For example, if a proximal portion of the endovascular tool advances 10 mm and the distal tip only advances 2 mm, then buckling of the endovascular tool is occurring within the body. In some embodiments, buckling may occur under the insertion force if resistance is encountered. This typically occurs when the distal tip of the endovascular tool is being inserted against the vessel wall or encountering resistance. Buckling can also occur when the distal tip of the endovascular tool is unobstructed, but the endovascular tool is navigating distally or through tortuous paths.

Buckling can be problematic for several reasons. For example, the more buckling that is present, the less predictable the motion of the endovascular tool behavior becomes. In particular, when buckling occurs, what happens at the distal tip of the endovascular tool no longer accurately reflects the commands given by the operator. Buckling also poses a safety concern. Buckling leads to a buildup of force within the endovascular tool, similar to a compressed spring. While resistance (such as friction with the vessel wall) prevents insertion, that resistance may suddenly give way, causing rapid, uncontrolled forward movement of the distal tip of the endovascular tool. This can result in perforation, which can be life-threatening. Accordingly, eliminating or reducing buckling can be important.

The robotic medical system 100 described herein can be configured to implement buckling reduction processes. For example, when inserting endovascular tools from a stationary position, unless there is a specific user preference (applicable in only a small number of situations), all internal buckling should be removed before insertion. This optimizes control of the distal tip of the endovascular tool and ensures the safest method of advancing the tool. To manage buckling, when endovascular tools are being inserted, the absolute forward motion of the distal tip should never be less than threshold distance compared to the forward motion of the helm or proximal end of the endovascular tool. If this occurs, the user should be alerted and insertion stopped immediately.

The buckling reduction processes of the robotic medical system 100 can include retracting the endovascular tool the system detects that the distal tip of the endovascular tool moves backwards. When buckling is present, the distal tip should remain stationary while retracting. Once the buckling is removed, the distal tip will start to move backwards, indicating that the buckling has been removed from the system.

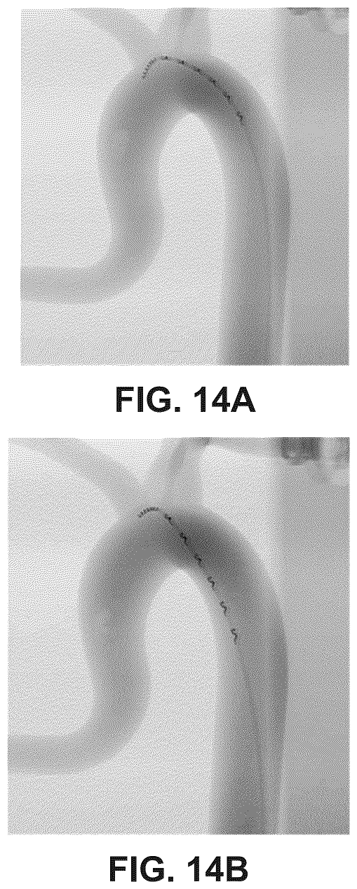

FIG. 14A illustrates an example X-ray image of a distal tip of an endovascular tool positioned within a vessel. In FIG. 14A a degree of buckling is present as indicated by the significant bowing at the distal end of the endovascular instrument. This buckling creates a danger as it represents energy stored in the endovascular tool that could be released suddenly, like a spring, causing the distal tip of the endovascular tool to rapidly and unpredictably advance. Accordingly, such buckling should be removed. FIG. 14B illustrates the same endovascular tool within the vessel after a degree of the buckling has been removed. For example, one can see that much of the bowing shape at the distal end of the endovascular tool has been removed.

As noted above, buckling can be removed by retracting the endovascular tool until the distal tip moves backwards. Retracting the endovascular tool until the tip moves in any direction may not be sufficient to remove buckling because, as the buckling is removed, in some instances, the tip may move forward or the tip may rotate as the endovascular tool straightens. In either case, the system should continue to retract the endovascular tool to further remove buckling. Accordingly, the buckling removal processes described herein can involve retracting the endovascular instrument until the distal tip is seen moving backwards.

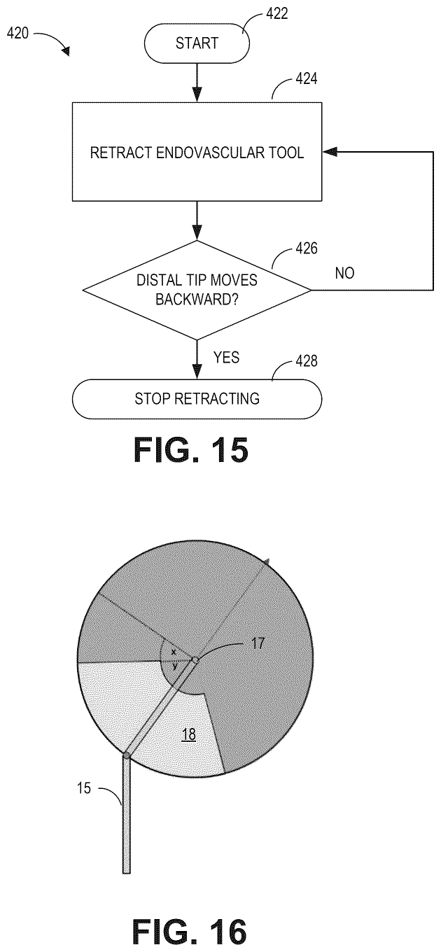

FIG. 15 illustrates an example method 420 for removing buckling in a robotic medical system. The method 420 can manage buckling that occurs within the body when a proximal end of the endovascular tool advances farther than the distal tip of the endovascular tool moves forward.

The method 420 begins at block 422. In some embodiments, block 422 is triggered based on a user input. For example, a user may provide an input to begin the buckling removal process. In other embodiments, buckling may be managed automatically, and the robotic medical system may automatically begin the buckling removal process when no movement commands are provided by the user. The buckling removal process can be performed automatically as a background task when the enable input described previously is active, but no motion commands are provided.

Once the method 420 begins, at block 424, the endovascular tool can begin to be retracted using endovascular robot 102. The method 420 moves to decision state 426 while the endovascular robot 102 continues to retract the endovascular tool. At decision state 426, the system evaluates whether the distal tip of the endovascular tool moves backward. Backwards motion of the distal tip of the endovascular tool can be determined using computer vision analysis of an X-ray image captured by the medical imager. If no sufficient backward motion of the distal tip of the endovascular tool is detected, the method 420 returns to block 424 and continues to retract the endovascular tool. If, at decision state 426, a sufficient backwards motion of the distal tip is detected, the method 420 moves to block 428 and stops retracting the endovascular tool.

The backwards motion of the distal tip of the endovascular tool detected at decision state 426 can be a movement greater that exceeds a predetermined threshold indicative of the removal of buckling. As described herein, the ability of the robotic medical system to sense the tool tip position using computer vision allows the system to detect when the tip moves sufficiently backward and then stop retracting. This capability also allows us to track the distance moved by the distal tip of the endovascular tool.

In the method 420 and other buckling removal processes described herein, backwards motion of the distal tip of the endovascular tool can be defined in several ways. For example, in one embodiment, the backwards direction is defined relative to the X-ray image. For example, any movement in a downward direction on the X-ray can be considered backward motion. This, however, may be problematic in some instances as the endovascular tool may not always be advanced in an upward direction relative to the X-ray. In some instances, for example, as the distal tip of the endovascular instrument traverses through the aortic arch, the distal tip may naturally move downward.

In some embodiments, “backwards” can be defined relative to the current heading of the endovascular instrument. For example, during the buckling removal process, the endovascular tool can be retracted until the distal tip is detected moving by a threshold distance in a direction opposite to the current endovascular tool heading. In some embodiments, the endovascular moves backwards until the distal tip moves backwards in a direction that is opposite the current heading (plus or minus 90 degrees) for distance that is equal to 1% of the image. Other threshold distances can be used. If the distal tip is detected move in a different direction, e.g., not in the direction opposite the current heading (plus or minus 90 degrees) by a distance greater than the threshold (e.g., 1% of the image), then the position and heading measurements are recalculated.

FIG. 16 illustrates that the endovascular instrument 15 can be retracted until the distal tip 17 moves into an arcuate area 18 defined in a direction opposite the current heading of the endovascular instrument. In the example of FIG. 16, the arcuate region is defined by angle x and a distance y. The angle x can be defined by a line perpendicular to the current heading of the endovascular toll and the start of the arcuate area. In some embodiments, the angle X can be between 0 and 45 degrees. The distance y can be a length (e.g., measured in mm, pixels, or a percentage of the length of the image) In some embodiments, the distance y can be between 10-50 pixels. In some embodiments, the distance y can be between 0.5 and 4 mm. In some embodiments, the distance can be a percentage of the image between 0.1% and 5%.

### Automated Tool Tip Tracking and X-Ray Masking for Visualization and X-Ray Reduction

The automated identification of a tool, and parts of a tool such as the tip, may be leveraged to optimize control of a decoupled imaging system or patient table. For example, movement of a C-arm (in the case of endovascular procedures) or a camera (in the case of laparoscopic surgery) could be precisely controlled/centered on the tool in question without manual operation. In addition, understanding of the tool position can also be leveraged to mask regions of the x-ray not required for visualization and therefore significantly reduce radiation. This can be extremely useful for the following reasons. For example, automated tool tip tracking can improve image optimization. Knowledge of the tool position enables automated centering of the tool in the image which results in the ideal procedural view. Additionally or alternatively, automated tool tip tracking can improve the speed and efficiency of procedures. For example, when the medical imager automatically tracks the endovascular tool, the operator does not need to divert attention to moving the medical imager through the procedure. Further, automated tool tip tracking can provide for resource optimization. That is, it can free up another member of staff to do something other than control a camera or x-ray. Finally, automated tool tip tracking can provide for radiation reduction. For example, leveraging tool pose and understanding of the anatomy can allow for masking of the regions of the image not required. By masking these regions and concentrating the image on the point of interest the robotic medical system can significantly reduce radiation.

Automated tool tip tracking can be extremely useful for interventional/surgical applications where the source of input imaging is decoupled from the navigating or interventional tool. In these procedures an assistant must manually track the surgeon's tools. Invariably this is done with a degree of lag and imprecision. For example, during an angiography procedure, automatic tool tip tracking can maintain the tool tip halfway across and one third up the screen the entire time. The physician must drop tools and manually readjust the screen to focus on the tool.

### X-Ray Detector to Enable Motion

In some embodiments, the robotic medical system can include an X-ray detector (e.g., a Geiger counter or similar device that is configured to measure the presence of X-rays). The Geiger counter can be positioned, for example, on the endovascular robot 102, bed mount 104, bed 116, or elsewhere in the operating environment where it will be exposed to X-rays from the medical imager 110.

The X-ray detector can be used to detect when the medical imager 110 is active. A signal from the X-ray detector can be used by the robotic medical system 100 as an input such that the robotic medical system 100 can only allow for forward movement of an endovascular tool when the medical imager is activated.

### Controlling Small Caliber Tools in a Robotic Medical System

This section describes systems and methods for control of small caliber endovascular tools, such as guidewires, microcatheters, and the like. The small caliber endovascular tools can be coupled to a robotic medical system that can facilitate movement (e.g., one or more of insertion, retraction, articulation, and roll, depending on the type of tool).

Because of the small diameter of such endovascular tools, fine control can be difficult, as the tools can be generally of limited rigidity (e.g., flimsy or floppy) and subject to high frictional forces (e.g., frictional forces of adjacent/nested tools or frictional forces of the vessel through which the tools are traversing). For these reasons, user commands may not produce the desired motion commanded by the operator or the system.

In some embodiments, the methods and systems for the control of small caliber tools can be applied to and/or facilitate automated removal of a guidewire or microcatheter or management of slack/buckling before insertion of the tool. In some embodiments, the methods and systems for the control of small caliber tools can include automated friction removal or reduction maneuvers, especially during insertion, including vibration, oscillation and back/forth motion of the tool. Additionally, in some embodiments, the methods and systems for the control of small caliber tools can be applied to and/or facilitate prevention of unsafe insertion by noting a discrepancy between tip movement and helm movement. For example, if the tool tip does not advance as expected given an insertion of the tool, this can be detected and addressed.

There are optimal ways to control small caliber endovascular tools (e.g. guidewires, microcatheters). Typically, optimal ways to control small caliber endovascular tools have included optimizing the physical characteristics of the guidewire, particularly with regard to its relationship with the surrounding tools. For example, often, the guidewire should be at least 0.0025″ smaller in outer diameter (OD) than the surrounding tool. Optimal ways to control small caliber endovascular tools have also included ensuring that the guidewire is constantly moistened and lubricated by a continuous, slow flow of saline or Heparinized Saline.

This section discusses new mechanisms that can be employed to facilitate control of small caliber endovascular tools. This can include, for example, optimizing the speed of guidewire insertion. For example, an amount from which a commanded motion varies from an actual motion can be determined, and the speed at which the tool is moved can be controlled (e.g., decreased or increased) to minimize the degree of variance.

As another example, the methods and system for optimizing guidewire control can include eradicating or reducing guidewire internal slack and buckling (by automation or otherwise). For example, in some embodiments, guidewire insertion should only begin once the slack and buckling have been (automatically or not) removed to a point where any further retraction would cause the wire to retract. In this regard, a degree of slack or buckling can be determined and minimized before additional commanded motion is provided.

As another example, the methods and system for optimizing guidewire control can include minimizing friction between the guidewire, surrounding tools, and the vessel wall through guidewire vibration, oscillation, back and forth motion, and/or oscillation and back and forth motion of the tool surrounding the guidewire. For example, as a tool is inserted or retracted, the tool can also be vibrated, oscillated, moved with small back and forth motions, or the like to minimize friction effects.

As another example, the methods and system for optimizing guidewire control can include preventing guidewire insertion against resistance, which can be defined, for example, by a discrepancy of more than 1.5 cm between the tip motion and the proximal end of the guidewire within the first 2 cm of proximal guidewire insertion. For example, if an insertion is provided at a proximal end of the tool and results in an insertion at the distal end of the tool that varies from the proximal end insertion by an amount exceeding a threshold, the system can be configured to stop providing commanded motion until these issues are resolved.

In some instances, optimal guidewire control can be a control scheme that satisfies one or more of the following user needs: (1) when the user sends a command to move the guidewire forward by x-cm, the guidewire tip moves forward x-cm (or by a corresponding amount that is within a predetermined threshold); (2) the guidewire does not move forward in an uncontrolled fashion (e.g., without a user-initiated command); (3) when the user sends a command to roll the guidewire x degrees, the guidewire rolls x degrees (or by a corresponding amount that is within a predetermined threshold); (4) the guidewire does not roll in an uncontrolled fashion (e.g., without a user initiated command), nor does it roll more than +−180 degrees; (5) guidewire roll is easy and intuitive and approximates how the user would rotate a guidewire with their hand; (6) it is easy to set guidewire roll back to 0 degrees. In some embodiments, one or more of these objectives is achieved with the methods and systems described herein.

For example, there may be a number of actions to meet the requirements noted above. These include can include minimizing guidewire friction. Guidewires may experience friction with other tools they are inserted into, such as microcatheters and/or the vessel wall. There are several ways to reduce this friction, including vibrating the guidewire, oscillating the guidewire, the tool it is inserted into or both in a limited range of motion (e.g. plus or minus 22.5 degrees.), inserting and retracting the guidewire or the tool it is inserted into in an oscillating motion of, for example, plus or minus 2 mm.

Additionally, actions to meet the requirements noted above can include minimizing guidewire internal slack and buckling. For example, guidewires should not be moved if there is a substantial amount of internal slack or buckling present. This can cause the guidewire to dangerously and uncontrollably jump, and it can also build up force in the body of the stationary guidewire. Ideally, a guidewire should be inserted from a stationary position with all internal slack and buckling removed. In some embodiments, forward motion of the tip should not exceed a 1.5 cm discrepancy compared to the forward motion of the proximal end.

Further, it is possible to optimize the physical characteristics of the guidewire, including using guidewires with soft/atraumatic tips, optimizing for pushability, apply lubricious coatings, sizing the guidewire appropriately in relation to surrounding tools (e.g., the wire should always be at least 0.0025″ smaller than the inner diameter of the tool it travels through).

In some embodiments, the methods and systems provide that it is not needed to rotate the tip of the guidewire more than plus or minus 180d. For example, if the distal tip of the guidewire cannot be rotated to its ideal position by rotating the proximal end of the guidewire between −180 degrees to +180 degrees, then the distal tip of the guidewire cannot be safely rotated into its ideal position.

### Automated Incline Control

In some embodiments, the incline of a robotically controlled surgical tool or instrument can be controlled. For example, in some embodiments, the incline can be controlled in an automated manner. A robotic medical system can be configured to automatically control the incline of a tool to be parallel to a vessel's centerline. The centerline of a vessel can be determined from a three-dimensional model of the patient's vasculature obtained from a pre-operative scan of the patient. The point on the vessel centerline can be chosen by determining where the tip of the catheter is. Automated control in some embodiments may utilize a co-registered anatomical model to determine the incline of the vessel's centerline at the tools location. For example, registration and tool position on the x-ray image can provide an estimate of where the tool tip is in the vessel anatomy (with an unknown depth dimension). This predicted position can be used to find the closest vessel centerline.

In some embodiments, automatic incline control can be toggled off and/or the incline commands can be provided manually. If toggled off or provided manually, a user will explicitly control heading and incline independently. When controlling heading, and incline is less than 45 degrees, the system can be configured to hold incline at its current position. When incline is greater than 45 degrees, the target heading will be applied with 45 degrees as incline target, so the catheter moves in the desired direction.

### Additional Details

It will now be evident to those skilled in the art that there has been described herein methods, systems, and devices for improved routing of catheters and other devices to targeted anatomical locations using robotically controlled assemblies. Although the inventions hereof have been described by way of several embodiments, it will be evident that other adaptations and modifications can be employed without departing from the spirit and scope thereof. The terms and expressions employed herein have been used as terms of description and not of limitation; and thus, there is no intent of excluding equivalents, but on the contrary, it is intended to cover any and all equivalents that may be employed without departing from the spirit and scope of the inventions.

While the disclosure has been described with reference to certain embodiments, it will be understood that various changes may be made, and equivalents may be substituted for elements thereof without departing from the scope of the disclosure. In addition, many modifications will be appreciated to adapt a particular instrument, situation, or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above but should be determined only by a fair reading of the claims that follow.

While the embodiments disclosed herein are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the recited order. The methods disclosed herein include certain actions taken by a practitioner; however, they can also include any third-party instruction of those actions, either expressly or by implication. For example, actions such as “advancing a catheter or microcatheter” or “advancing one portion of the device (e.g., linearly) relative to another portion of the device to rotate the distal end of the device” include instructing advancing a catheter” or “instructing advancing one portion of the device,” respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

The various illustrative logical blocks, modules, routines, and algorithm steps described in connection with the embodiments disclosed herein can be implemented as electronic hardware, or as a combination of electronic hardware and executable software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a similarity detection system, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A similarity detection system can be or include a microprocessor, but in the alternative, the similarity detection system can be or include a controller, microcontroller, or state machine, combinations of the same, or the like configured to estimate and communicate prediction information. A similar detection system can include electrical circuitry configured to process computer-executable instructions. Although described herein primarily with respect to digital technology, a similarity detection system may also include primarily analog components. For example, some or all of the prediction algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a similarity detection system, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An illustrative storage medium can be coupled to the similarity detection system such that the similarity detection system can read information from, and write information to, the storage medium. In the alternative, the storage medium can be integral to the similarity detection system. The similarity detection system and the storage medium can reside in an ASIC. The ASIC can reside in a user terminal. In the alternative, the similarity detection system and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A robotic medical system, comprising:
an endovascular robot configured at robotically manipulate one or more endovascular instruments insertable into a vasculature of a patient during an endovascular procedure;
a user control station configured to receive:
robot control user inputs, provided by a user, for causing the endovascular robot to robotically manipulate the one or more endovascular instruments, and
an enable input provided by the user; and
a robot control unit in communication with the user control station and the endovascular robot, the robot control unit comprising at least one processor and at least one computer memory, the at least one computer memory comprising computer-readable instructions that, when executed by the processor, cause the processor to:
cause the endovascular robot to manipulate the one or more endovascular instruments according to the robot control user inputs received from the user control station only when an enable input is also received from the user control station; and
cause the endovascular robot to perform automated background tasks when the enable input is received from the user control station and no robot control user inputs are received from the user control station.

2. The robotic medical system of claim 1, wherein the computer-readable instructions, when executed by the processor, further cause the processor to disregard the robot control user inputs received from the user control station when no enable input is received from the user control station.

3. The robotic medical system of claim 2, wherein the automated background tasks comprise performing a slack removal process configured to remove slack from the one or more endovascular instruments.

4. The robotic medical system of claim 3, wherein the slack removal process comprises causing retraction, by a proximally located helm of the endovascular robot, of the one or more endovascular instruments until a force or torque measured at a distally located helm of the endovascular robot exceeds a predetermined force or torque threshold.

5. The robotic medical system of claim 4, wherein the predetermined force or torque threshold is determined so as to be indicative of the one or more endovascular instruments going taut between the proximally located helm and the distally located helm.

6. The robotic medical system of claim 5, wherein the automated background tasks comprise performing a buckling removal process configured to remove buckling from the one or more endovascular instruments.

7. The robotic medical system of claim 6, wherein the buckling removal process comprises causing, with the endovascular robot, retraction of the one or more endovascular instruments until a computer vision analysis of a medical image showing a distal tip of the one more endovascular instruments determines that the distal tip moves in a backwards direction relative to a current heading of the distal tip by a distance that exceeds a predetermined threshold.

8. The robotic medical system of claim 7, wherein the backwards direction relative to the current heading comprises an arcuate area.

9. The robotic medical system of claim 1, wherein the automated background tasks comprise adjusting a position of a moveable helm of the endovascular robot in a manner that does not cause movement of a distal tip of the one or more endovascular instruments.

10. The robotic medical system of claim 1, wherein user control station comprises a foot pedal, and the enable input is provided through the foot pedal.

11. A robotic medical method comprising:
receiving, from a user control station one or more of:
robot control user inputs, provided by a user, for causing an endovascular robot to robotically manipulate one or more endovascular instruments, or
an enable input provided by the user;
causing the endovascular robot to manipulate the one or more endovascular instruments according to the robot control user inputs received from the user control station only when an enable input is also received from the user control station; and
causing the endovascular robot to perform automated background tasks when the enable input is received from the user control station and no robot control user inputs are received from the user control station.

12. The robotic medical method of claim 11, further comprising disregarding the robot control user inputs received from the user control station when no enable input is received from the user control station.

13. The robotic medical method of claim 12, wherein the automated background tasks comprise performing a slack removal process configured to remove slack from the one or more endovascular instruments.

14. The robotic medical method of claim 13, wherein the slack removal process comprises causing retraction, by a proximally located helm of the endovascular robot, of the one or more endovascular instruments until a force or torque measured at a distally located helm of the endovascular robot exceeds a predetermined force or torque threshold.

15. The robotic medical method of claim 14, wherein the predetermined force or torque threshold is determined so as to be indicative of the one or more endovascular instruments going taut between the proximally located helm and the distally located helm.

16. The robotic medical method of claim 15, wherein the automated background tasks comprise performing a buckling removal process configured to remove buckling from the one or more endovascular instruments.

17. The robotic medical method of claim 16, wherein the buckling removal process comprises causing, with the endovascular robot, retraction of the one or more endovascular instruments until a computer vision analysis of a medical image showing a distal tip of the one more endovascular instruments determines that the distal tip moves in a backwards direction relative to a current heading of the distal tip by a distance that exceeds a predetermined threshold.

18. The robotic medical method of claim 17, wherein the backwards direction relative to the current heading comprises an arcuate area.

19. The robotic medical method of claim 11, wherein the automated background tasks comprise adjusting a position of a moveable helm of the endovascular robot in a manner that does not cause movement of a distal tip of the one or more endovascular instruments.

20. The robotic medical method of claim 11, wherein user control station comprises a foot pedal, and the method further comprises receiving the enable input from the foot pedal.
